SEQUENCE LISTING (4 sequences)

SEQ ID NO: 1 - DNA, 53 bp, Artificial Sequence
  Description of Artificial Sequence Synthetic primer
tgtgggtcttggtgttttccatagaagatttttggatgcattctttgtaggtg

SEQ ID NO: 2 - DNA, 57 bp, Artificial Sequence
  Description of Artificial Sequence Synthetic primer
tgcggaataatcattcttgaccaaaccctatggtttcttccacggccaatgttagct

SEQ ID NO: 3 - DNA, 23 bp, Artificial Sequence
  Description of Artificial Sequence Synthetic primer
ggcgtggttaacgtccgcgtggg

SEQ ID NO: 4 - DNA, 22 bp, Artificial Sequence
  Description of Artificial Sequence Synthetic primer
ccctatggtttcttccacggcc

(19) United States
(12) Patent Application Publication
(10) Pub. No.: US 2020/0375996 A1
(43) Pub. Date: Dec. 3, 2020

(54) METHOD OF TREATING NEURODEGENERATIVE DISORDERS BY RESCUING ALPHA-SYNUCLEIN TOXICITY

(71) Applicants: D.E. Shaw Research, LLC, New York, NY (US); WHITEHEAD INSTITUTE FOR BIOMEDICAL RESEARCH, Cambridge, MA (US)

(72) Inventors: Yibing SHAN, Millburn, NJ (US); Venkat MYSORE, South Orange, NJ (US); Susan LINDQUIST, Cambridge, MA (US); Dan TARDIFF, Arlington, MA (US); Srividya CHANDRAMOULI, Somerville, MA (US)

(73) Assignees: D.E. Shaw Research, LLC, New York, NY (US); WHITEHEAD INSTITUTE FOR BIOMEDICAL RESEARCH, Cambridge, MA (US)

(21) Appl. No.: 16/062,502

(22) PCT Filed: Dec. 14, 2016
(86) PCT No.: PCT/US2016/066687

§ 371 Date: Jun. 14, 2018

Related U.S. Application Data
(60) Provisional application No. 62/267,698, filed on Dec. 15, 2015.

(51) Int. Cl. A61K 31/5377; A61K 31/496; A61P 25/00; A61K 31/53

(52) U.S. Cl. CPC A61K 31/5377; A61K 31/53; A61P 25/00; A61K 31/496

(57) ABSTRACT

A method for treating neurodegenerative disease in a subject in need thereof by administering to the subject an effective amount of a Nedd4 activator as described herein.

### CROSS REFERENCE TO RELATED APPLICATION

The present application is a U.S. National Stage application under 35 U.S.C. § 371 of International Patent Application No. PCT/US16/66687, filed Dec. 14, 2016, which claims the benefit of U.S. Provisional Patent Application No. 62/267,698, filed Dec. 15, 2015, the contents of which are hereby incorporated by reference.

### SEQUENCE LISTING

The instant application contains a Sequence Listing which has been submitted electronically in ASCII format and is hereby incorporated by reference in its entirety. Said ASCII copy, created on Jun. 29, 2020, is named 2206689_00126US2_SL.txt and is 1,326 bytes in size.

### FIELD

This application relates the treatment of neurodegenerative diseases, such as Parkinson's disease, Alzheimer's disease, or Lewy body disease by administering an effective amount of a compound disclosed herein. Also disclosed herein are methods of modulating α-synuclein toxicity or E3 ubiquitin ligase in a subject in need thereof by administering to the subject an effective amount of a compound disclosed herein.

### BACKGROUND

There is a need for successful disease-modifying therapies against common and progressive neurodegenerative diseases (ND), such as Parkinson's Disease (PD) and Alzheimer's Disease (AD). Modeling the cellular pathologies that underlie α-synucleinopathies (including PD) in yeast recapitulates the derangements in protein trafficking and mitochondrial dysfunction that are seen in neurons and PD patients. The ease of yeast culture and the robust growth phenotypes induced by α-synuclein greatly facilitate high-throughput compound screening. While phenotypic screens are unbiased, the formidable challenge of deciphering mechanisms of Action (MOA) can limit the advancement of lead compounds by impeding target-guided medicinal chemistry and early clinical evaluation of on-target efficacy. Therefore, there is a need to identify compounds that address underlying cellular pathologies in NDs and to define the specific target space in which they act.

### SUMMARY

In one aspect, the present application provides a method for treating neurodegenerative disease in a subject in need thereof, the method comprising administering to the subject an effective amount of a Nedd4 activator as disclosed herein.

In accordance with another aspect, the present application provides a method of modulating α-synuclein toxicity in a subject in need thereof, the method comprising administering to the subject an effective amount of a Nedd4 activator as disclosed herein.

In yet another aspect, the present application discloses a method of modulating E3 ubiquitin ligase in a subject in need thereof, the method comprising administering to the subject an effective amount of a Nedd4 activator as disclosed herein.

A method for treating neurodegenerative disease in a subject in need thereof, the method comprising administering to the subject an effective amount of a Nedd4 activator as disclosed herein is also presented in this application.

In still another aspect, a method for treating a neurodegenerative disease associated with α-synuclein toxicity in a subject in need thereof is disclosed herein. The method comprises administering to the subject an effective amount of a compound as disclosed herein.

In another aspect, the present invention provides a pharmaceutical composition comprising at least one compound as described herein and a pharmaceutically-acceptable carrier or diluent.

In yet another aspect, the present invention provides a method for treating a psychotic disorder in a mammalian species in need thereof, the method comprising administering to the mammalian species a therapeutically effective amount of at least one compound as described herein, wherein the compound comprises a Nedd4 activator that promotes Nedd4-dependent Golgi to vacuole or plasma membrane to vacuole trafficking of adaptor protein Sna3.

In a further another aspect, the present invention provides a method for treating a neurodegenerative disorder in a mammalian species in need thereof, the method comprising administering to the mammalian species a therapeutically effective amount of at least one compound as described herein, wherein the neurodegenerative disorder is selected from Parkinson's disease, Alzheimer's disease, and Lewy body disease. The compounds disclosed herein can also be used to treat other synucleinopathies such as multiple system atrophy and pure autonomic failure.

### BRIEF DESCRIPTION OF THE FIGURES

FIG. 1A shows: Left panel is the structure of previously identified NAB and the predicted binding site of NAB with the Rsp5 HECT domain hinge region. The right panel shows compound ‘32’, which was predicted to bind to this same site based on an in silico screen of 2 million compounds. Compound structures are distinct and binding to Rsp5 appears similar, yet distinct, as well.

FIG. 1B. shows dose-response curves of α-synuclein-expressing yeast treated with increasing concentrations of both NAB2 and ‘32’. Efficacy increases to a peak around 10 μM and then NAB2/′32′ begin to slow growth, most likely due to over activation of Rsp5.

FIG. 1C. shows Western blot analysis of a protein trafficking substrate—Cpy—that is differentially cleaved when trafficking from the Endoplasmic Reticulum to the Golgi and Vacuole. Accumulation of the high molecular weight band reflects a block in vesicle trafficking. Both NAB and ‘32’ ameliorate this defect.

FIG. 2 provides representative dose-response curves of sample compounds showing some activity in rescuing α-synuclein toxicity in yeast. X-axis is compound concentration in μM and Y-axis is rescue normalized to maximal rescue by NAB2. FIG. 2 Upper right provides structure of starting hit ‘32’ and potent analog ‘2877’. Lower left, structures of effective compounds that are less toxic to cells and do not have bell-shaped curve. Lower right, structures of compounds that have very modest activity against α-synuclein toxicity.

FIGS. 3A, 3B, and 3C show that NAB and ‘32’ both promote K63-linked ubiquitination of proteins in a Nedd4-dependent manner. FIG. 3A provides results of an assay designed show that NAB2 treatment causes an increase in K63 pUB in human iPS derived from neuronal cultures.

NAB2 mediated increase is dependent primarily upon Nedd4 as shown in FIG. 3B, wherein the assay was performed on human iN neurons.

NAB2 mediated increase is dependent primarily upon Nedd4 as shown in FIG. 3C, wherein the assay was performed on cells from the HEK-293 cell line.

FIG. 4 shows dose-response curves of α-synuclein-expressing yeast treated with increasing concentrations of various compounds disclosed herein relative to ‘32’.

FIGS. 5A-5B show binding curves of NAB2 binding to Rsp5. Back Scattering Interferometry (BSI) assay technology was used to obtain binding measurements. FIG. 5A shows the binding of NAB2 to Rsp5 as a function of concentration of NAB2 on a logarithmic scale. FIG. 5B shows the binding of NAB2 to Rsp5 as a function of concentration of NAB2. The dissociation constant (K_{d}) was determined to be 0.84±0.13 μM (R²=0.92).

FIGS. 6A-6B show binding curves of DES-005212 binding to Rsp5. BSI assay technology was used to obtain binding measurements. FIG. 6A shows the binding of DES-005212 to Rsp5 as a function of concentration of DES-005212 on a logarithmic scale. FIG. 6B shows the binding of DES-005212 to Rsp5 as a function of concentration of DES-005212. The dissociation constant (K_{d}) was determined to be 0.68±0.18 μM (R²=0.81).

FIGS. 7A-7B show binding curves of DES-002877 binding to Rsp5. BSI assay technology was used to obtain binding measurements. FIG. 7A shows the binding of DES-002877 to Rsp5 as a function of concentration of DES-002877 on a logarithmic scale. FIG. 6B shows the binding of DES-002877 to Rsp5 as a function of concentration of DES-002877. The dissociation constant (K_{d}) was determined to be 1.7±0.4 μM (R²=0.86).

FIGS. 8A-8B show the effect of compounds on rescue of aSyn toxicity in yeast.

FIG. 8A shows the effect of NAB and NAB29 on rescue of aSyn toxicity in yeast. The effect of doxorubicin (positive control) and DMSO (negative control) are also shown. FIG. 8B shows the effect of DES-2179, DES-4114, DES-2877, DES-2966, NAB2, DES-2184, DES-4109, DES-2997, and DMSO on rescue of aSyn toxicity in yeast. DES-2877 and DES-4144 were most effective in rescuing aSyn toxicity in yeast. DES-2866 and DES-2184 were also effective in rescuing aSyn toxicity in yeast.

FIGS. 9A-9B show toxicity profiles of compounds on WT control yeast strain. FIG. 9A shows the toxicity profiles of NAB2, DES-2179, DES-4109, DES-2184, DES-2866, DES-2877, and DES-4114 on WT control yeast strain. FIG. 9B shows the toxicity profiles of NAB29, DES-4145, DES-4106, DES-2764, DES-2997, DES-3001, and DES-4117 on WT control yeast strain. Compounds that were active in rescuing synuclein all showed toxicity to some extent. DES-4114 was the least toxic among active analogs, and also the most effective in rescuing aSyn toxicity. Inactive compounds were not toxic in WT yeast cells.

FIG. 10 shows aSyn-expressing yeast cells treated with DMSO, NAB2, DES-2877 (“2877”), and DES-4114 (“4114”). Morphological analysis shows that rescue of aSyn toxicity by DES-2877 and DES-4114 is accompanied by an accumulation of vesicular intermediates in yeast cells.

FIG. 11A shows transport pathways from the yeast late Golgi to the vacuole. Sna3-GFP is an Rsp5 adaptor protein that relies on ubiquitination for its MVB sorting. Direct Binding to Rsp5 Mediates Ubiquitin-independent Sorting of Sna3 via the Multivesicular Body (MVB) Pathway. Sna3p undergoes Rsp5-dependent polyubiquitylation, with K63-linked Ub chains. FIG. 11B shows the effect of compounds on ubiquitination of Sna3-GFP in WT and α-syn cells. DES-2877 and DES-4114 cause an increase in the polyubiquitinated Sna3-GFP. FIG. 11C shows the ratio of Sna3-GFP to free GFP for various compounds in WT and α-syn cells. GFP is cleaved from Sna3-GFP upon reaching the vacuole and is a measure of its MVB sorting. FIG. 11D shows the effect of compounds on Carboxypeptidase Y (CPY) trafficking intermediates enroute to the vacuole. DES-2877 and DES-4114 cause an increase in accumulation of CPY trafficking intermediates en route to the vacuole. CPY bound to its receptor (Vps10p) leaves the late Golgi in clathrin-coated vesicles, which fuse with the PVC. In the PVC, the ligand/receptor complex dissociates, and CPY is transported to the vacuole. CPY processing is an indication of MVB sorting and turnover and may indicate an increase in TGN-MVB trafficking compared to MVB-vacuole trafficking rate.

FIGS. 12A-12B show toxicity profiles of compounds on rat cortical neurons. FIG. 12A shows the toxicity profiles of DES-2184, DES-2179, DES-4114, DES-2877, and DES-2866. FIG. 12B shows the toxicity profiles of DES-4117, DES-4109, DES-3001, DES-2997, and DES-2764. The compounds that were active in rescuing aSyn were toxic in rat cortical neurons. The less effective compounds were less toxic. 24 hour time point showed identical trends.

FIG. 13A shows immunoblot analysis of the ability of various compounds to induce K63-Ub linkages. FIG. 13B shows changes in the abundance of different ubiquitin chain linkages HEK-293 cells in response to treatment with various compounds.

FIG. 14A shows a heatmap representation of aSyn toxicity rescue for various sample compounds. The heatmap shows the percent change in OD600 as compared to untreated yeast cells expressing alpha-synuclein. FIG. 14B shows the EC₄₀ and IC₄₀ values for selected compounds represented in FIG. 14A.

FIG. 15A shows a schematic of Sna3-GFP endosomal trafficking to the vacuole, where GFP is cleaved. FIGS. 15B-15F show Western blot analyses of Sna3-GFP in cells treated with various compounds.

FIGS. 16A-16F show the effect of treatment with different compounds (at 10 μM) in a Sna3-GFP ubiquitination assay.

### DETAILED DESCRIPTION

### Definitions

The following are definitions of terms used in the present specification. The initial definition provided for a group or term herein applies to that group or term throughout the present specification individually or as part of another group, unless otherwise indicated.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. The term “(C₁-C₆)alkyl” refers to a straight or branched chain alkane (hydrocarbon) radical containing from 1 to 6 carbon atoms, such as n-hexyl, 2-methylpentyl, 3-methylpentyl, 4-methylpentyl, 2,3-dimethylbutyl, 2,2-dimethylbutyl, in addition to those exemplified for “(C₁-C₄)alkyl.” “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂R₃, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The terms “halogen” or “halo” refer to chlorine, bromine, fluorine or iodine.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of a compound of the present invention may be formed, for example, by reacting a compound I with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecyl sulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

Compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% pure (“substantially pure” compound I), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

### Compounds

In another aspect, the present invention provides a pharmaceutical composition comprising at least one compound as described herein and a pharmaceutically-acceptable carrier or diluent.

### Utility and Methods of Use

In certain embodiments, this invention provides a use of at least one compound as described herein in the manufacture of a medicament for treating a disorder or treating a neurodegenerative disease associated with α-synuclein toxicity. The compounds disclosed herein may be used to reduce alpha-synuclein toxicity in a cell (e.g., neuron or glial cell) or subject. The compounds disclosed herein may be used for reducing, inhibiting, or preventing α-synuclein toxicity.

The compounds of the present can be used to modulate α-synuclein toxicity in a subject in need thereof by administering to the subject an effective amount of a Nedd4 activator as disclosed herein.

In certain embodiments, the compounds disclosed herein can be used to modulate E3 ubiquitin ligase in a subject by administering to the subject an effective amount of a Nedd4 activator as disclosed herein.

In view of the utility of the compounds according to the invention, there is provided a method of treating warm-blooded animals, including humans, suffering from any one of the diseases mentioned hereinbefore, and a method of preventing in warm-blooded animals, including humans, any one of the diseases mentioned hereinbefore.

Said methods comprise the administration, i.e., the systemic or topical administration, preferably oral administration, of a therapeutically effective amount of a compound according to the invention to warm-blooded animals, including humans.

Therefore, the invention also relates to a method for the prevention and/or treatment of any one of the diseases mentioned hereinbefore comprising administering a therapeutically effective amount of compound according to the invention to a patient in need thereof.

In accordance with one aspect, a method for treating neurodegenerative disease in a subject in need thereof is disclosed. The method comprises administering to the subject an effective amount of a Nedd4 activator of formula (I) or (IA).

In accordance with another aspect, the present application provides a method of modulating α-synuclein toxicity or modulating E3 ubiquitin ligase in a subject in need thereof, wherein the method comprises administering to the subject an effective amount of a Nedd4 activator of formula (I).

Compounds of formula (I) and (IA) are represented by the following structures:

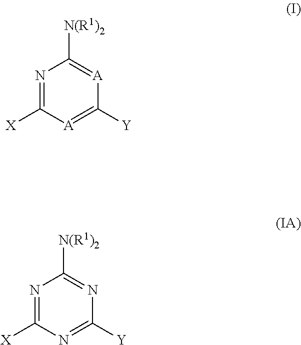

wherein
A is independently CH or N;
R¹ is independently H, (C₁-C₄)-alkyl, phenyl, or each R¹ together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl;

### X is

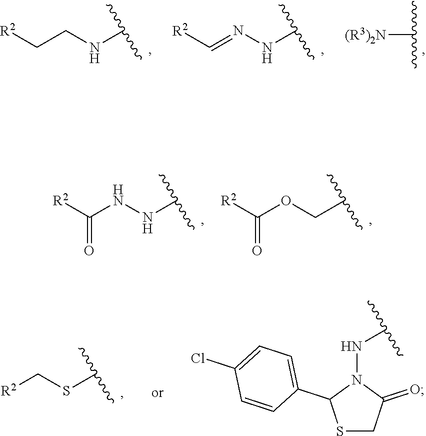

### Y is

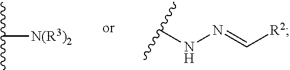

R² is independently phenyl, benzyl, naphthyl, furanyl, indolyl, pyridinyl, pyrazinyl, pyrimidinyl, or thiophenyl, wherein said phenyl, benzyl, naphthyl, furanyl, indolyl, pyridinyl, pyrazinyl, pyrimidinyl, or thiophenyl is optionally independently substituted with one or more H, (C₁-C₄)-alkyl, ((C₁-C₄)-alkyl)OH, OH, O—(C₁-C₄)-alkyl, CF₃, halogen, S—(C₁-C₄)-alkyl, S(O)(C₁-C₄)-alkyl, OC(O)CH₃, OC(O)Ph, OCH₂Ph, OCH₂CO₂H, OCH₂CN, CN, N((C₁-C₄)-alkyl)₂, morpholin-4-yl, or Ph(CO₂H), or is

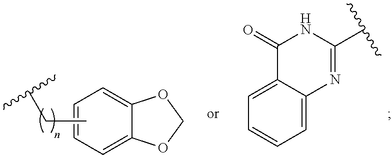

R³ is independently H, (C₁-C₄)-alkyl, phenyl, benzyl, or naphthyl, wherein said phenyl, benzyl, or naphthyl is optionally independently substituted with one or more H, (C₁-C₄)-alkyl, OH, O—(C₁-C₄)-alkyl, CF₃, or halogen, or is (C₁-C₄)-alkyl and each (C₁-C₄)-alkyl together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl, or is

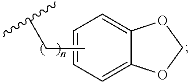

R⁴ is H or (C₁-C₃)-alkyl; and n is independently 0 or 1.

In certain embodiments, X is

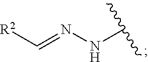

### Y is

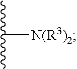

and
R¹ is (C₁-C₄)-alkyl, wherein each R¹ together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl.

In some embodiments, each R¹ together with the nitrogen to which they are attached form NR⁴-piperazine, piperidine, pyrrolidine, azetidine, or morpholine.

In certain embodiments, each R¹ together with the nitrogen to which they are attached form morpholine.

In certain embodiments, X is

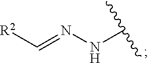

### Y is

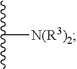

and
R² is phenyl or pyridinyl, wherein said phenyl or pyridinyl is optionally independently substituted with one or more H, (C₁-C₄)-alkyl, OH, O—(C₁-C₄)-alkyl, CF₃, halogen, S—(C₁-C₄)-alkyl, OC(O)CH₃, OC(O)Ph, OCH₂Ph, OCH₂CO₂H, OCH₂CN, CN, N((C₁-C₄)-alkyl)₂, morpholin-4-yl, or Ph(CO₂H).

In some cases, R² is phenyl or pyridine-4-yl, wherein said phenyl or pyridine-4-yl is optionally independently substituted with one or more H, (C₁-C₄)-alkyl, OH, O—(C₁-C₄)-alkyl, CF₃, halogen, OCH₂CN, or N((C₁-C₄)-alkyl)₂.

In certain embodiments, R¹ is (C₁-C₄)-alkyl, wherein each R¹ together with the nitrogen to which they are attached form NR⁴-piperazine, piperidine, pyrrolidine, azetidine, or morpholine.

In certain embodiments, X is

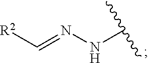

### Y is

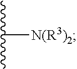

and
R³ is independently H, phenyl, or naphthyl, wherein said phenyl or naphthyl is optionally independently substituted with one or more H, (C₁-C₄)-alkyl, CF₃, or halogen.

In certain embodiments, R² is phenyl or pyridine-4-yl, wherein said phenyl or pyridine-4-yl is optionally independently substituted with one or more H, (C₁-C₄)-alkyl, OH, O—(C₁-C₄)-alkyl, CF₃, halogen, OCH₂CN, or N((C₁-C₄)-alkyl)₂; and

R¹ is (C₁-C₄)-alkyl, wherein each R¹ together with the nitrogen to which they are attached form NR⁴-piperazine, piperidine, pyrrolidine, azetidine, or morpholine.

In certain embodiments, X is

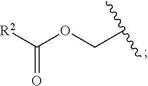

### Y is

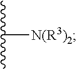

and
R² is phenyl, pyridinyl, or pyrazinyl, wherein said phenyl, pyridinyl, or pyrazinyl, is optionally independently substituted with one or more (C₁-C₄)-alkyl, ((C₁-C₄)-alkyl)OH, OH, O—(C₁-C₄)-alkyl, or S(O)(C₁-C₄)-alkyl.

In particular embodiments, each X and Y is independently

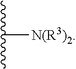

In certain embodiments, each X and Y is independently

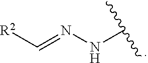

In certain embodiments, X is

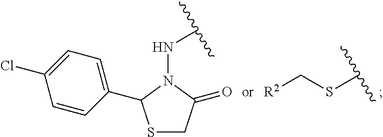

and

### R² is

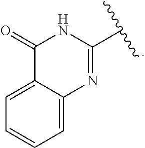 [SMILES: *c1nc2ccccc2c(=O)[nH]1]

In certain embodiments, the Nedd4 activator is selected from the group consisting of:

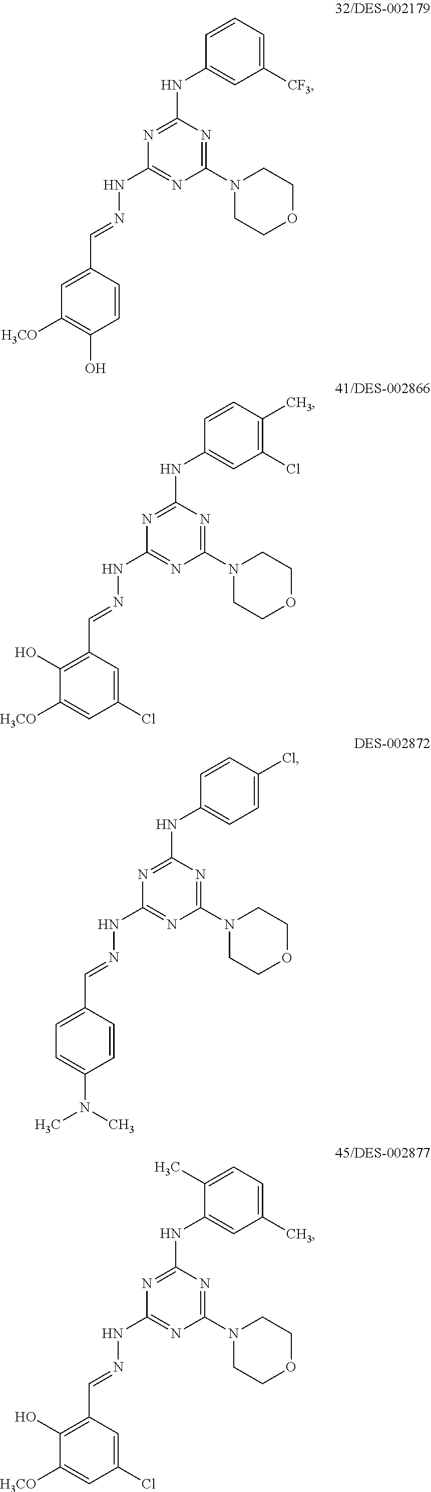 [SMILES: COc1cc(/C=N/Nc2nc(Nc3cccc(C(F)(F)F)c3)nc(N3CCOCC3)n2)ccc1O ; COc1cc(Cl)cc(/C=N/Nc2nc(Nc3ccc(C)c(Cl)c3)nc(N3CCOCC3)n2)c1O ; CN(C)c1ccc(/C=N/Nc2nc(Nc3ccc(Cl)cc3)nc(N3CCOCC3)n2)cc1 ; COc1cc(Cl)cc(/C=N/Nc2nc(Nc3cc(C)ccc3C)nc(N3CCOCC3)n2)c1O]

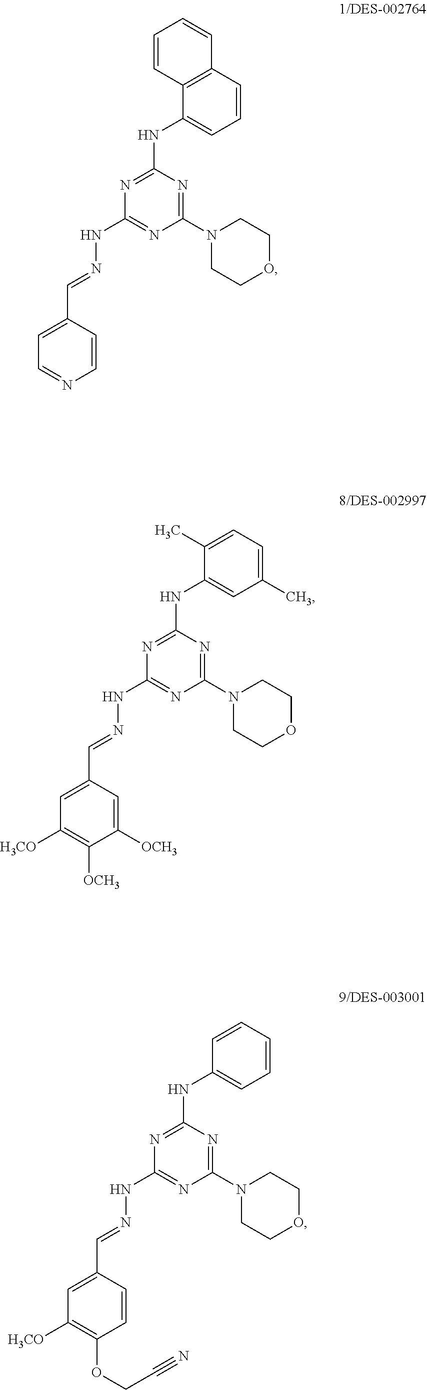 [SMILES: C(=N\Nc1nc(Nc2cccc3ccccc23)nc(N2CCOCC2)n1)/c1ccncc1 ; COc1cc(/C=N/Nc2nc(Nc3cc(C)ccc3C)nc(N3CCOCC3)n2)cc(OC)c1OC ; COc1cc(/C=N/Nc2nc(Nc3ccccc3)nc(N3CCOCC3)n2)ccc1OCC#N]

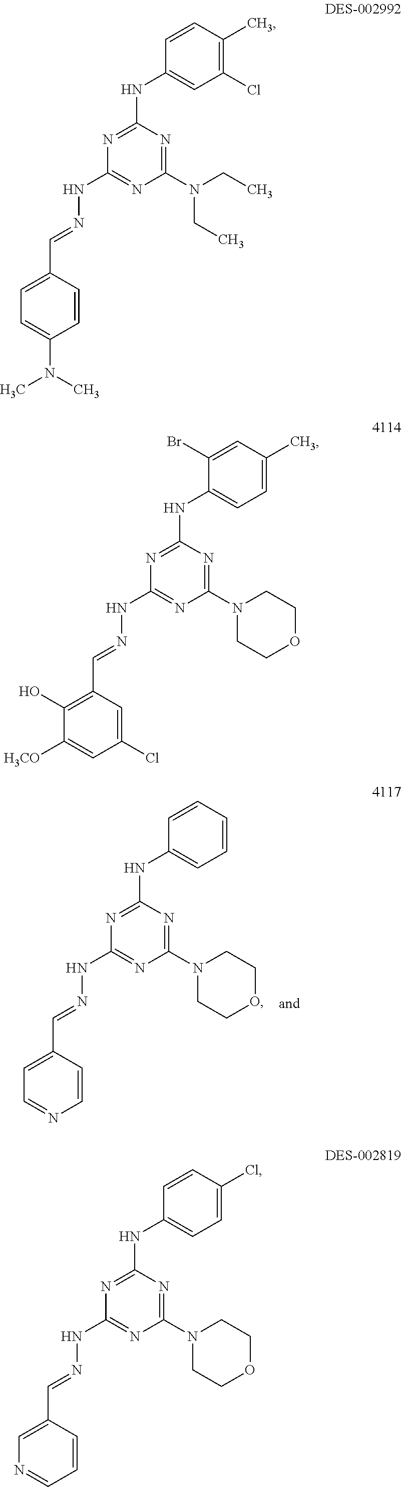 [SMILES: CCN(CC)c1nc(N/N=C/c2ccc(N(C)C)cc2)nc(Nc2ccc(C)c(Cl)c2)n1 ; COc1cc(Cl)cc(/C=N/Nc2nc(Nc3ccc(C)cc3Br)nc(N3CCOCC3)n2)c1O ; C(=N\Nc1nc(Nc2ccccc2)nc(N2CCOCC2)n1)/c1ccncc1 ; Clc1ccc(Nc2nc(N/N=C/c3cccnc3)nc(N3CCOCC3)n2)cc1]

or a pharmaceutically acceptable salt thereof, or a pharmaceutical composition thereof.

In certain embodiments, the Nedd4 activator modulates ubiquitin-mediated endosomal transport. In other embodiments, the Nedd4 activator increases ubiquitination or polyubiquitination. In some cases, the increase in ubiquitination or polyubiquitination comprises modulating E3 ubiquitin ligase.

The Nedd4 activator may promote Nedd4-dependent Golgi to vacuole or plasma membrane to vacuole trafficking of adaptor protein Sna3. In some cases, the Nedd4 activator promotes Nedd4-dependent endocytosis of leucine permease.

The present application is also directed to a method for treating neurodegenerative disease in a subject in need thereof, wherein the method comprises administering to the subject an effective amount of a Nedd4 activator of formula (II):

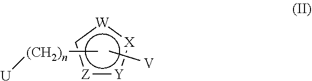

wherein
each of W, X, Y, Z is independently O, S, NR⁶, N, C, or CR⁷;
at least one of W, X, Y, Z must be O, S, NR⁶, or N;
R⁶ is independently H, (C₁-C₃)alkyl, phenyl;
R⁷ is independently H, (C₁-C₃)alkyl, or phenyl;
n is an integer from 0-3;
U is OR⁸, SR⁸, (SO₂)R⁸, (SO₂)NR⁸, N(R⁸)₂, NH(CO)R⁸, NHCH₂R⁸, phenyl, or

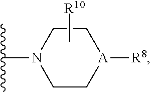

or

### U is

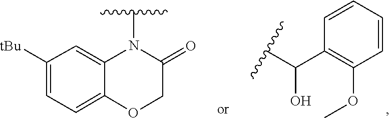 [SMILES: *N1C(=O)COc2ccc(C(C)(C)C)cc21 ; *C(O)c1ccccc1OC]

### or U is,

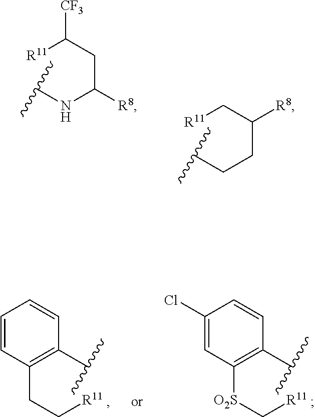

R⁸ is phenyl, naphthyl, pyridinyl, pyrimidinyl, pyrazinyl, quinolinyl, isoquinolinyl or benzothiazolyl, wherein said phenyl, naphthyl, pyridinyl, pyrimidinyl, pyrazinyl, quinolinyl, isoquinolinyl, or benzothiazolyl is optionally independently substituted with one or more H, (C₁-C₄)-alkyl, OH, O—(C₁-C₄)-alkyl, OCF₃, CF₃, halogen, CO₂((C₁-C₄)-alkyl), NH(CO)((C₁-C₄)-alkyl), (C₁-C₄)-alkyl((CO)NH₂), S—(C₁-C₄)-alkyl, triazole, or R⁸ is

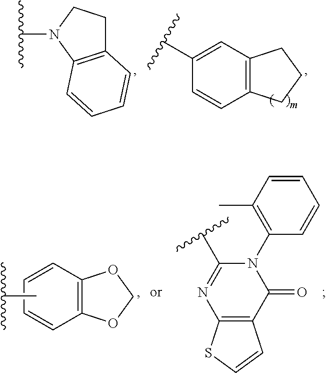

m is 1 or 2;

### V is

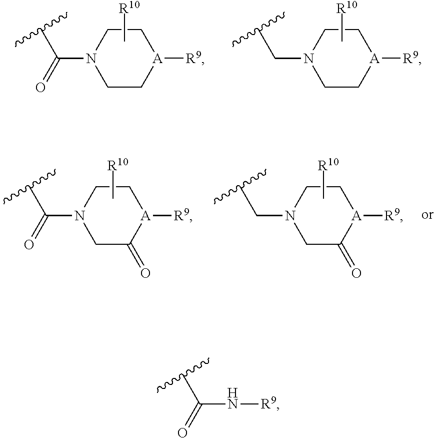

R⁹ is phenyl, pyridinyl, pyrimidinyl, or pyrazinyl, wherein said phenyl, pyridinyl, pyrimidinyl, or pyrazinyl is optionally independently substituted with one or more H, (C₁-C₄)-alkyl, —OH, —O—(C₁-C₄)-alkyl, —CF₃, halogen, —CN, —C(O)((C₁-C₄)-alkyl), or R⁹ is —CH₂CH₂N((C₁-C₄)-alkyl)₂;
A is independently CH, N, or C(OH);
R¹⁰ is H or (C₁-C₄)-alkyl; and
R¹¹ is H or R¹¹ together with the carbon to which it is attached forms a 5-6 membered ring with W or Z.

In some cases, W is O;

each of Y and Z is CH;

### X is C;

n is 1; and

### V is

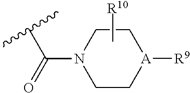

and is bonded to X.

In some embodiments, U is OR⁸, SR⁸, (SO₂)R⁸, (SO₂)NR⁸, N(R⁸)₂, NH(CO)R⁸, or

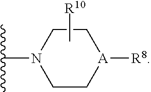

In some embodiments, R⁸ is phenyl, naphthyl, pyridinyl, pyrimidinyl, quinolinyl, isoquinolinyl or benzothiazolyl, wherein said phenyl, naphthyl, pyridinyl, pyrimidinyl, quinolinyl, isoquinolinyl, or benzothiazolyl is optionally independently substituted with one or more H, (C₁-C₄)-alkyl, OH, O—(C₁-C₄)-alkyl, OCF₃, CF₃, halogen, CO₂((C₁-C₄)-alkyl), NH(CO)((C₁-C₄)-alkyl), (C₁-C₄)-alkyl((CO)NH₂), S—(C₁-C₄)-alkyl, or triazole.

In some embodiments, R⁹ is phenyl, pyridinyl, pyrimidinyl, or pyrazinyl, wherein said phenyl, pyridinyl, pyrimidinyl, or pyrazinyl is optionally independently substituted with one or more H, (C₁-C₄)-alkyl, OH, O—(C₁-C₄)-alkyl, CF₃, halogen, or —CN; A is N; and

R¹⁰ is H or (C₁)-alkyl.

In some embodiments, the Nedd4 activator is:

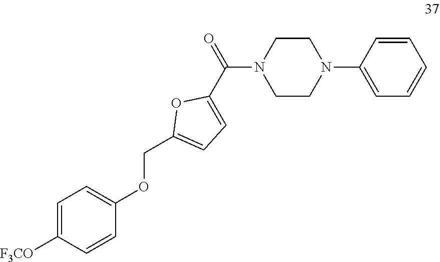 [SMILES: O=C(c1ccc(COc2ccc(OC(F)(F)F)cc2)o1)N1CCN(c2ccccc2)CC1]

In some embodiments, W is NR⁶;

each of X and Z is CH;

### Y is C;

n is 0;
U is (SO₂)R⁸;

### R⁸ is

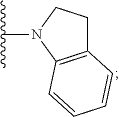 [SMILES: *N1CCc2ccccc21]

### V is

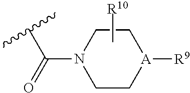

and is bonded to Y.
R⁹ is phenyl;

### A is N; and

### R¹⁰ is H.

In some embodiments, W is S;

### Z is N;

### X is C;

### Y is CR⁷;

R⁷ is H or CH₃;
n is 1;

### U is OR⁸;

R⁸ is phenyl, wherein said phenyl is substituted with CH₃ or halogen.

### V is

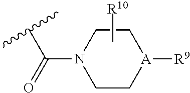

and is bonded to X;
R⁹ is phenyl or pyrimidinyl, wherein said phenyl or pyrimidinyl is optionally independently substituted with one or more H, (C₁-C₄)-alkyl, or halogen;

### A is N; and

### R¹⁰ is H.

In some embodiments, W is O;

each of X and Z is N;
Y is C and (CH₂)ₙ-U is bonded to Y;
n is 1;

### U is OR⁸;

R⁸ is phenyl, wherein said phenyl is substituted with CO₂((C₁-C₄)-alkyl), NH(CO)((C₁-C₄)-alkyl), or (C₁-C₄)-alkyl((CO)NH₂);

### V is

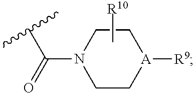

### A is N;

R⁹ is phenyl, wherein said phenyl is substituted with halogen; and

### R¹⁰ is H.

In certain embodiments, W is O;

### X is N;

### Y is C;

### Z is CR⁷;

### R⁷ is H;

n is 1;

### U is OR⁸;

R⁸ is phenyl, naphthyl, pyridinyl, quinolinyl, isoquinolinyl or benzothiazolyl, wherein said phenyl, naphthyl, pyridinyl, quinolinyl, isoquinolinyl, or benzothiazolyl is optionally independently substituted with one or more H, (C₁-C₄)-alkyl, OH, O—(C₁-C₄)-alkyl, S—(C₁-C₄)-alkyl, triazole, or

### R⁸ is

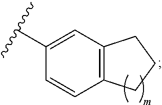

m is 2;

### V is

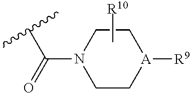

and is bonded to Y;
R⁹ is phenyl, pyridinyl, or pyrazinyl, wherein said phenyl, pyridinyl, or pyrazinyl is optionally independently substituted with one or more H, (C₁-C₄)-alkyl, —OH, or —C(O)((C₁-C₄)-alkyl);

### A is CH or N; and

R¹⁰ is H or CH₃.

In some embodiments, W is S;

### X is C;

each of Y and Z is CR⁷;
R⁷ is independently H or CH₃;
n is 1;

### U is OR⁸;

R⁸ is phenyl, wherein said phenyl is substituted with halogen;

### V is

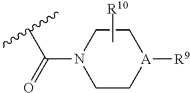

and is bonded to X;
R⁹ is pyrimidinyl;

### A is N; and

### R¹⁰ is H.

In some embodiments, W is S;

### X is C;

each of Y and Z is N;
n is 1;

### U is

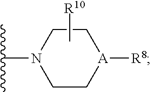

A is N or CH; and

R⁸ is phenyl, wherein said phenyl is substituted with OH or CH₃;

### V is

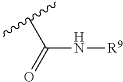

and is bonded to X;
R⁹ is phenyl, wherein said phenyl is substituted with (C₁-C₄)-alkyl or —O—(C₁-C₄)-alkyl; and

### R¹⁰ is H.

In some embodiments, W is O;

### X is CR⁷;

### R⁷ is H;

### Y is C;

### Z is N;

n is 1;
U is OR⁸, SR⁸, or

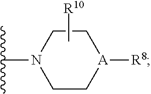

A is independently N;
R⁸ is phenyl, wherein said phenyl is substituted with O(C₁-C₄)-alkyl or halogen, or R⁸ is

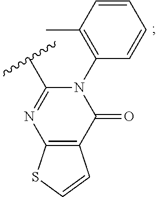 [SMILES: *c1nc2sccc2c(=O)n1-c1ccccc1C]

### V is

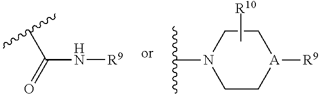

and is bonded to Y;
R⁹ is phenyl or pyridinyl, wherein said phenyl or pyridinyl is substituted with (C₁-C₄)-alkyl, —O—(C₁-C₄)-alkyl, or halogen; and

### R¹⁰ is H.

In some embodiments, W is NR⁶;

### X is N;

Y is C and (CH₂)ₙ-U is bonded to Y;

### Z is CR⁷;

### R⁶ is H;

### R⁷ is H;

n is 0 or 1;
   U is OR⁸ or (SO₂)NR⁸;
   R⁸ is phenyl, wherein said phenyl is substituted with —O—(C₁-C₄)-alkyl;

### V is

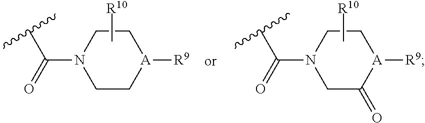

R⁹ is phenyl, pyridinyl, or pyrazinyl wherein said phenyl, pyridinyl, or pyrazinyl is substituted with (C₁-C₄)-alkyl or halogen;

### A is N; and

### R¹⁰ is H.

In some embodiments, W is NR⁶;

### Each of X and Z is N;

### Y is C;

R⁶ is phenyl;
n is 2;
U is phenyl;

### V is

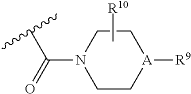

and is bonded to Y;
R⁹ is phenyl, wherein said phenyl is substituted with —O—(C₁-C₄)-alkyl;

### A is N; and

### R¹⁰ is H.

In some embodiments, W is N and (CH₂)ₙ-U is bonded to W;

each of X and Y is N;

### Z is C;

n is 1;

### U is

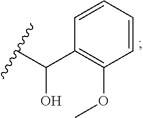 [SMILES: *C(O)c1ccccc1OC]

### V is

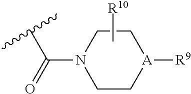

and is bonded to Z;
R⁹ is phenyl, wherein said phenyl is substituted with halogen;

### A is N; and

R¹⁰ is H.

In some embodiments, W is S;

### X is CR⁶;

### Y is C;

### Z is N;

### R⁶ is H;

n is 0 or 1;

### U is

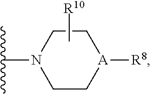

NH(CO)R⁸, or NHCH₂R⁸;
R⁸ is phenyl, wherein said phenyl is optionally substituted with one or more —O—(C₁-C₄)-alkyl or halogen, or R⁸ is

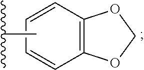

### V is

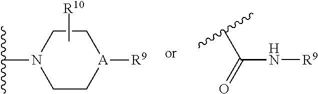

and is bonded to Y;
R⁹ is phenyl or pyridinyl, wherein said phenyl or pyridinyl is optionally substituted with halogen;
A is independently N or C(OH); and

### R¹⁰ is H.

In some embodiments, W is S;

each of X and Z is C;

### Y is CR⁶;

### R⁶ is H;

n is 0;

### U is

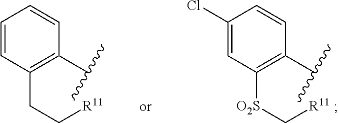

R¹¹ together with the carbon to which it is attached forms a 6 membered ring with Z; and

### V is

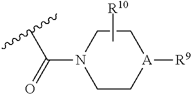

and is bonded to X.

In some embodiments, W is N;

### X is CR⁶;

### Y is C;

### Z is N;

### R⁶ is H;

n is 0;

### U is

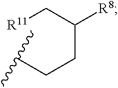

R¹¹ together with the carbon to which it is attached forms a 6 membered ring with W; and

### V is

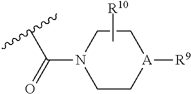

and is bonded to Y.

In some embodiments, W is N;

### X is N;

### Y is C;

### Z is CR⁶;

### R⁶ is H;

n is 0;

### U is

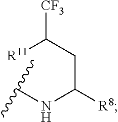

R¹¹ together with the carbon to which it is attached forms a 6 membered ring with W; and

### V is

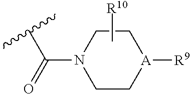

and is bonded to Y.

The present application provides a method for treating a neurodegenerative disease in a subject in need thereof, the method comprising administering to the subject an effective amount of a compound selected from the group consisting of:

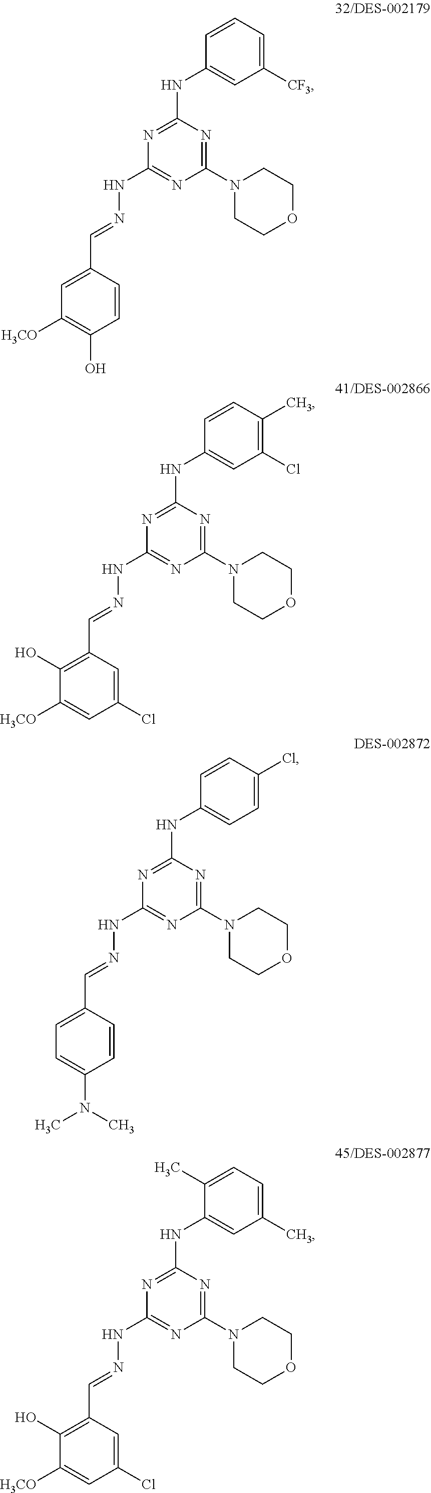 [SMILES: COc1cc(/C=N/Nc2nc(Nc3cccc(C(F)(F)F)c3)nc(N3CCOCC3)n2)ccc1O ; COc1cc(Cl)cc(/C=N/Nc2nc(Nc3ccc(C)c(Cl)c3)nc(N3CCOCC3)n2)c1O ; CN(C)c1ccc(/C=N/Nc2nc(Nc3ccc(Cl)cc3)nc(N3CCOCC3)n2)cc1 ; COc1cc(Cl)cc(/C=N/Nc2nc(Nc3cc(C)ccc3C)nc(N3CCOCC3)n2)c1O]

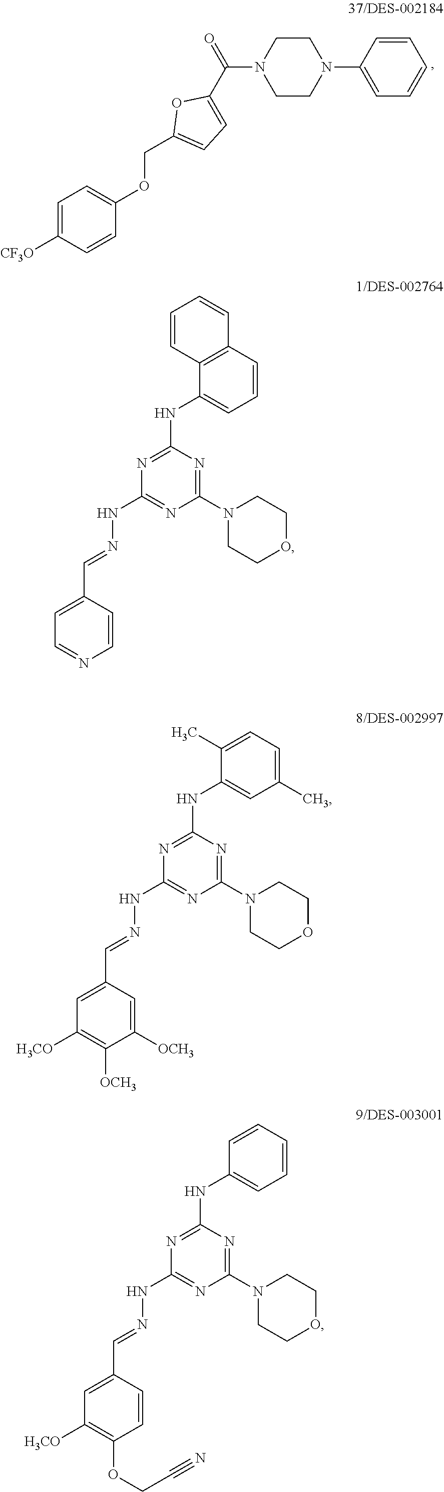

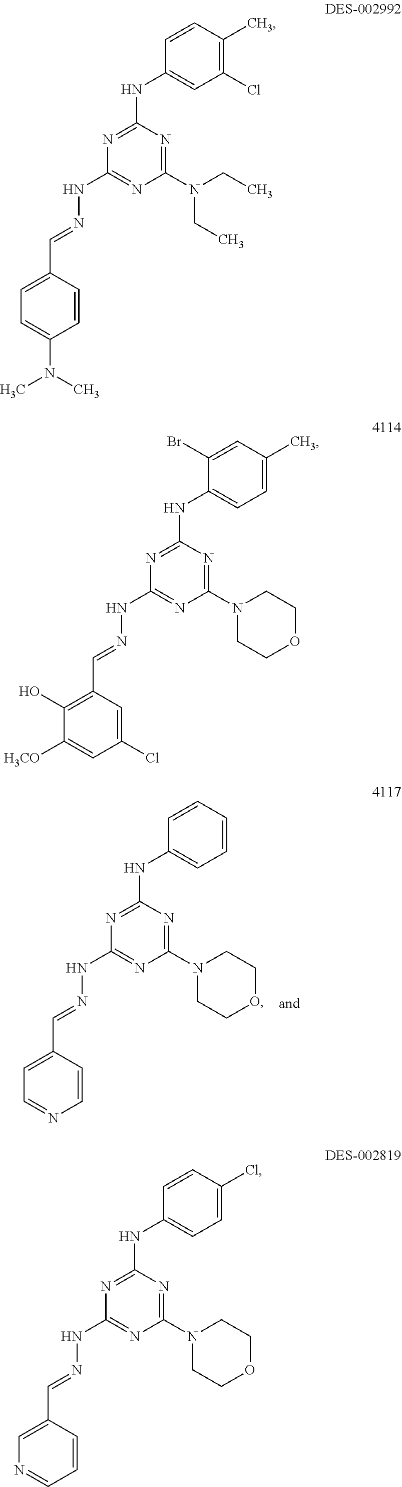 [SMILES: CCN(CC)c1nc(N/N=C/c2ccc(N(C)C)cc2)nc(Nc2ccc(C)c(Cl)c2)n1 ; COc1cc(Cl)cc(/C=N/Nc2nc(Nc3ccc(C)cc3Br)nc(N3CCOCC3)n2)c1O ; C(=N\Nc1nc(Nc2ccccc2)nc(N2CCOCC2)n1)/c1ccncc1 ; Clc1ccc(Nc2nc(N/N=C/c3cccnc3)nc(N3CCOCC3)n2)cc1]

or a pharmaceutically acceptable salt thereof, or a pharmaceutical composition thereof.

In accordance with another aspect, a method for treating a neurodegenerative disease associated with α-synuclein toxicity in a subject in need thereof is disclosed. The method comprises administering to the subject an effective amount of a compound selected from the group consisting of:

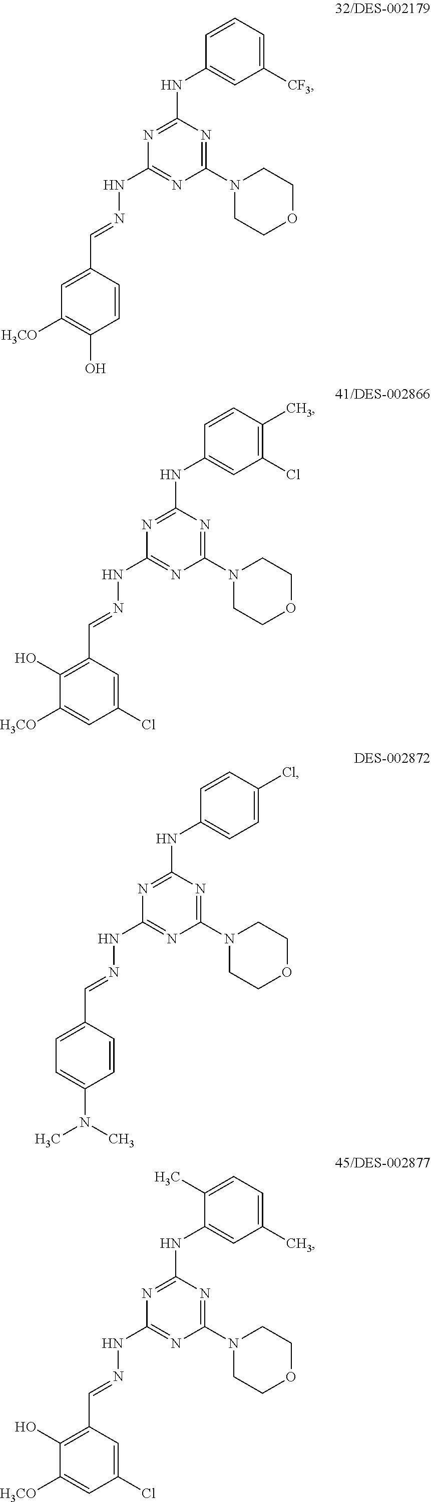 [SMILES: COc1cc(/C=N/Nc2nc(Nc3cccc(C(F)(F)F)c3)nc(N3CCOCC3)n2)ccc1O ; COc1cc(Cl)cc(/C=N/Nc2nc(Nc3ccc(C)c(Cl)c3)nc(N3CCOCC3)n2)c1O ; CN(C)c1ccc(/C=N/Nc2nc(Nc3ccc(Cl)cc3)nc(N3CCOCC3)n2)cc1 ; COc1cc(Cl)cc(/C=N/Nc2nc(Nc3cc(C)ccc3C)nc(N3CCOCC3)n2)c1O]

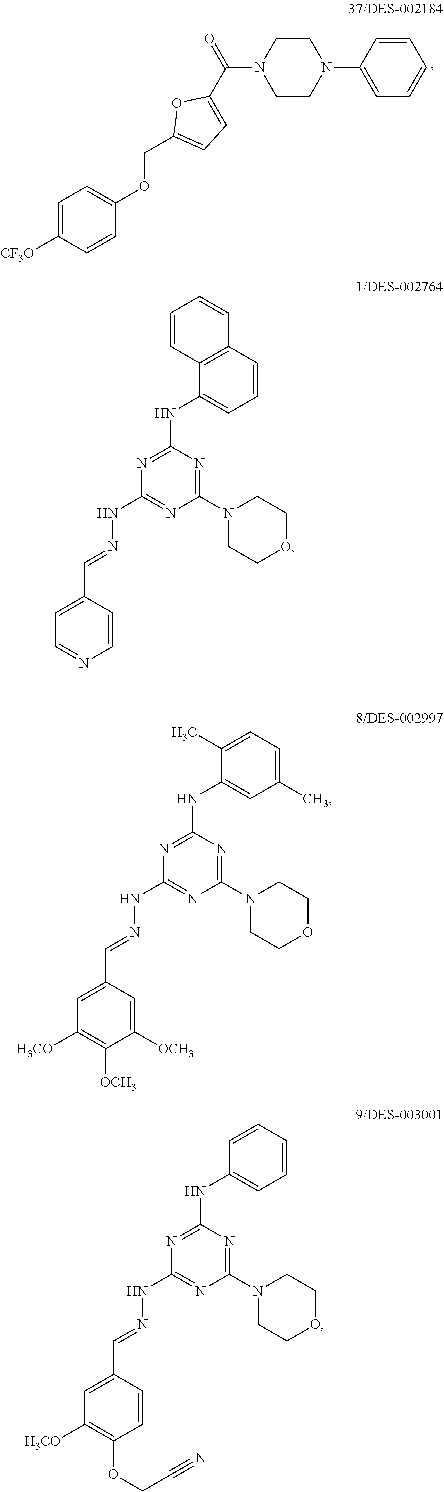

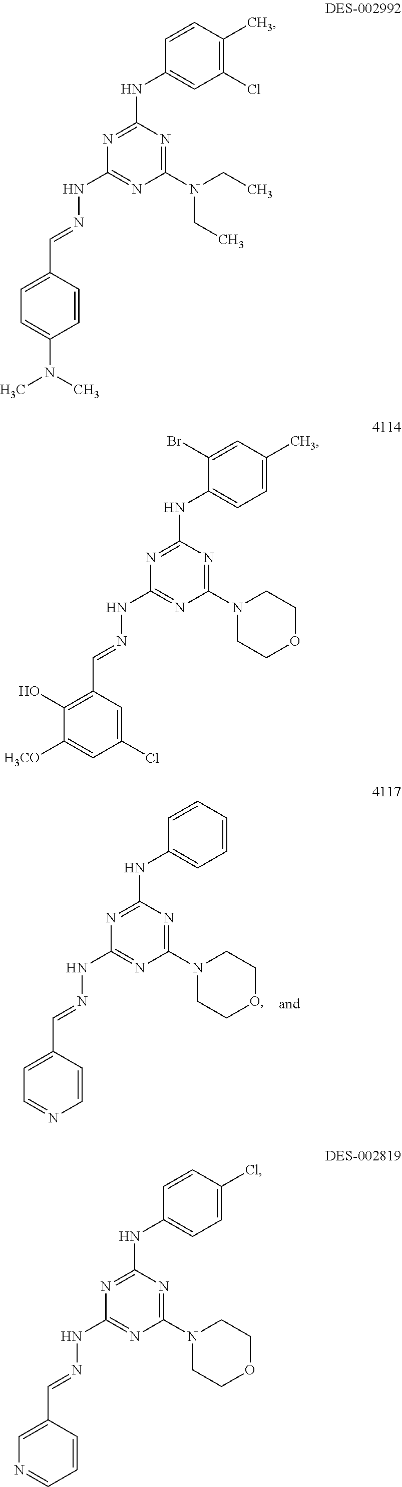 [SMILES: CCN(CC)c1nc(N/N=C/c2ccc(N(C)C)cc2)nc(Nc2ccc(C)c(Cl)c2)n1 ; COc1cc(Cl)cc(/C=N/Nc2nc(Nc3ccc(C)cc3Br)nc(N3CCOCC3)n2)c1O ; C(=N\Nc1nc(Nc2ccccc2)nc(N2CCOCC2)n1)/c1ccncc1 ; Clc1ccc(Nc2nc(N/N=C/c3cccnc3)nc(N3CCOCC3)n2)cc1]

or a pharmaceutically acceptable salt thereof, or a pharmaceutical composition thereof.

Specific examples of compounds useful in accordance with the present application include the compounds in Table 1 as well as pharmaceutically acceptable salt thereof, or a pharmaceutical composition thereof:

TABLE 1

| Series | Compound Structure | Compound Name |
|---|---|---|
| 32 | 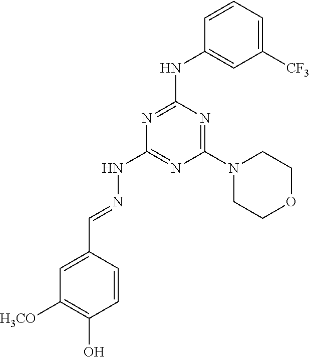 [SMILES: COc1cc(/C=N/Nc2nc(Nc3cccc(C(F)(F)F)c3)nc(N3CCOCC3)n2)ccc1O] | DES-2179; 32 |
| | 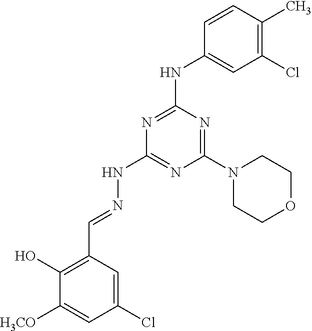 [SMILES: COc1cc(Cl)cc(/C=N/Nc2nc(Nc3ccc(C)c(Cl)c3)nc(N3CCOCC3)n2)c1O] | DES-2866; 41 |
| | 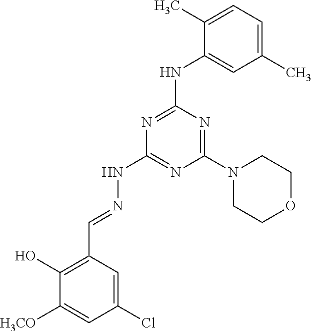 [SMILES: COc1cc(Cl)cc(/C=N/Nc2nc(Nc3cc(C)ccc3C)nc(N3CCOCC3)n2)c1O] | DES-2877; 45 |
| | 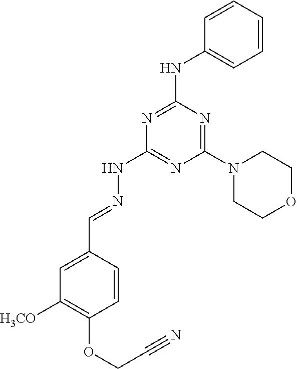 [SMILES: COc1cc(/C=N/Nc2nc(Nc3ccccc3)nc(N3CCOCC3)n2)ccc1OCC#N] | DES-3001; 9 |
| | 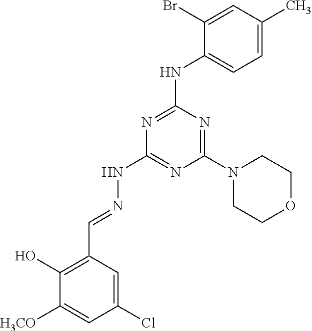 [SMILES: COc1cc(Cl)cc(/C=N/Nc2nc(Nc3ccc(C)cc3Br)nc(N3CCOCC3)n2)c1O] | DES-4114 |
| 4117 | 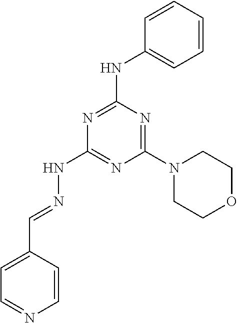 [SMILES: C(=N\Nc1nc(Nc2ccccc2)nc(N2CCOCC2)n1)/c1ccncc1] | DES-4117 |
| | 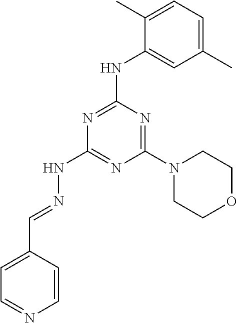 [SMILES: Cc1ccc(C)c(Nc2nc(N/N=C/c3ccncc3)nc(N3CCOCC3)n2)c1] | DES-5204 |
| | 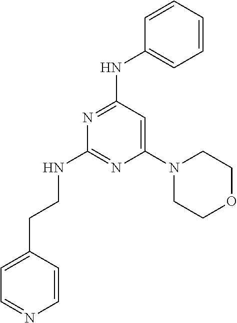 [SMILES: c1ccc(Nc2cc(N3CCOCC3)nc(NCCc3ccncc3)n2)cc1] | DES-5205 |
| | 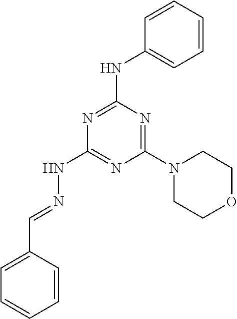 [SMILES: C(=N\Nc1nc(Nc2ccccc2)nc(N2CCOCC2)n1)/c1ccccc1] | DES-5208 |
| | 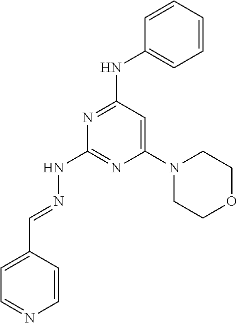 [SMILES: C(=N\Nc1nc(Nc2ccccc2)cc(N2CCOCC2)n1)/c1ccncc1] | DES-5210 |
| | 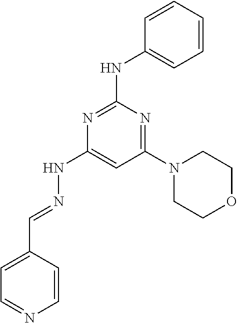 [SMILES: C(=N\Nc1cc(N2CCOCC2)nc(Nc2ccccc2)n1)/c1ccncc1] | DES-5212 |
| “37” | 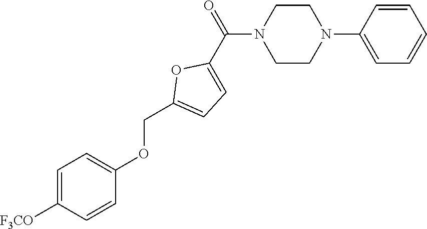 [SMILES: O=C(c1ccc(COc2ccc(OC(F)(F)F)cc2)o1)N1CCN(c2ccccc2)CC1] | DES-2184; 37 |
| | 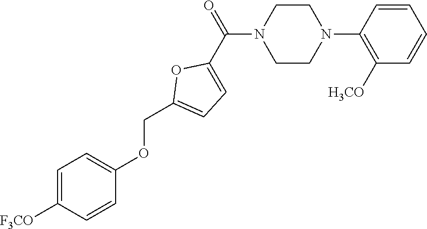 [SMILES: COc1ccccc1N1CCN(C(=O)c2ccc(COc3ccc(OC(F)(F)F)cc3)o2)CC1] | DES-2835 |
| | 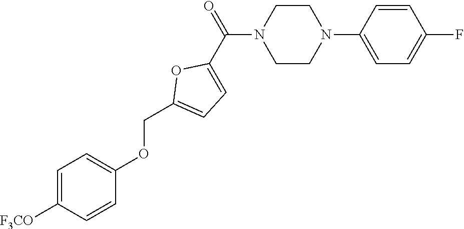 [SMILES: O=C(c1ccc(COc2ccc(OC(F)(F)F)cc2)o1)N1CCN(c2ccc(F)cc2)CC1] | DES-2842 |
| | 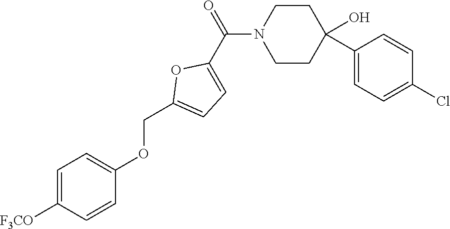 [SMILES: O=C(c1ccc(COc2ccc(OC(F)(F)F)cc2)o1)N1CCC(O)(c2ccc(Cl)cc2)CC1] | DES-2854 |
| | 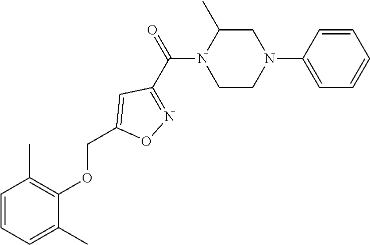 [SMILES: Cc1cccc(C)c1OCc1cc(C(=O)N2CCN(c3ccccc3)CC2C)no1] | DES-2868 |
| | 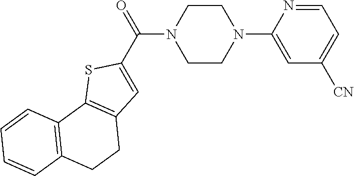 [SMILES: N#Cc1ccnc(N2CCN(C(=O)c3cc4c(s3)-c3ccccc3CC4)CC2)c1] | DES-2922 |
| | 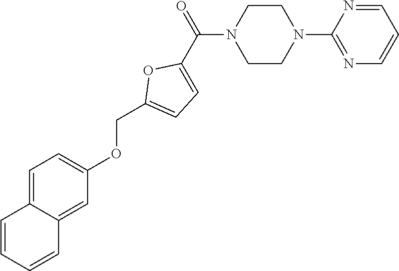 [SMILES: O=C(c1ccc(COc2ccc3ccccc3c2)o1)N1CCN(c2ncccn2)CC1] | DES-2926 |
| | 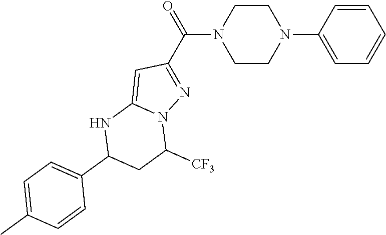 [SMILES: Cc1ccc(C2CC(C(F)(F)F)n3nc(C(=O)N4CCN(c5ccccc5)CC4)cc3N2)cc1] | DES-2960 |
| | 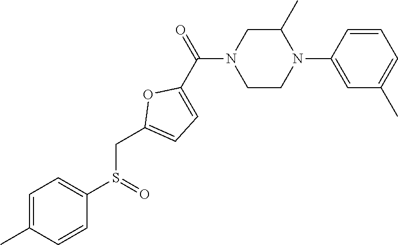 | DES-2977 |
| | 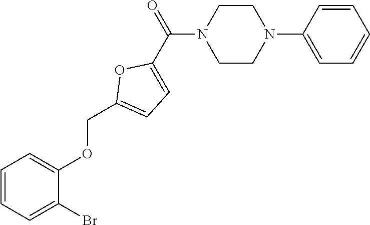 [SMILES: O=C(c1ccc(COc2ccccc2Br)o1)N1CCN(c2ccccc2)CC1] | DES-3026 |
| | 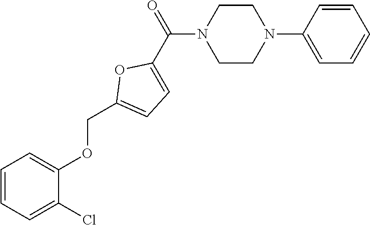 [SMILES: O=C(c1ccc(COc2ccccc2Cl)o1)N1CCN(c2ccccc2)CC1] | DES-3027 |
| | 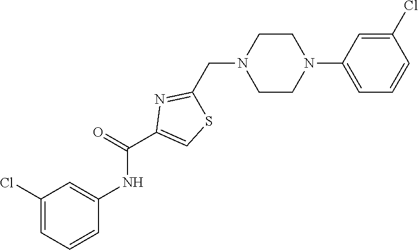 [SMILES: O=C(Nc1cccc(Cl)c1)c1csc(CN2CCN(c3cccc(Cl)c3)CC2)n1] | DES-3034 |
| | 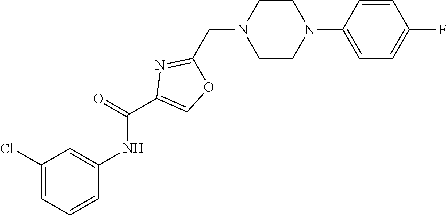 [SMILES: O=C(Nc1cccc(Cl)c1)c1coc(CN2CCN(c3ccc(F)cc3)CC2)n1] | DES-3035 |
| 28 | 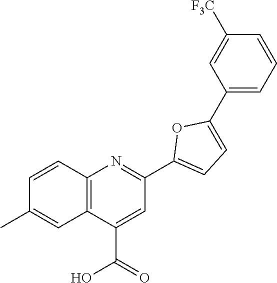 [SMILES: Cc1ccc2nc(-c3ccc(-c4cccc(C(F)(F)F)c4)o3)cc(C(=O)O)c2c1] | “28” |
| | 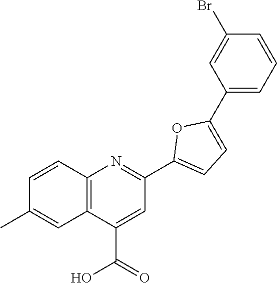 [SMILES: Cc1ccc2nc(-c3ccc(-c4cccc(Br)c4)o3)cc(C(=O)O)c2c1] | DES-2804 |
| | 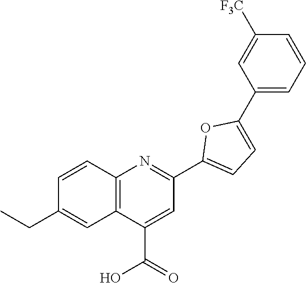 [SMILES: CCc1ccc2nc(-c3ccc(-c4cccc(C(F)(F)F)c4)o3)cc(C(=O)O)c2c1] | DES-2814 |
| | 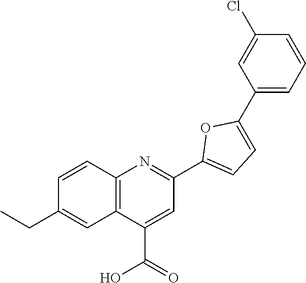 [SMILES: CCc1ccc2nc(-c3ccc(-c4cccc(Cl)c4)o3)cc(C(=O)O)c2c1] | DES-2815 |
| | 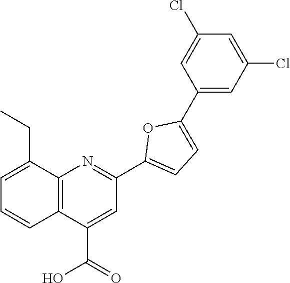 [SMILES: CCc1cccc2c(C(=O)O)cc(-c3ccc(-c4cc(Cl)cc(Cl)c4)o3)nc12] | DES-2816 |
| | 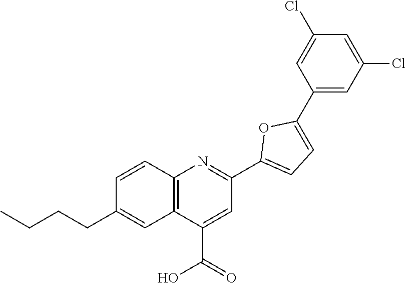 [SMILES: CCCCc1ccc2nc(-c3ccc(-c4cc(Cl)cc(Cl)c4)o3)cc(C(=O)O)c2c1] | DES-2817 |
| | 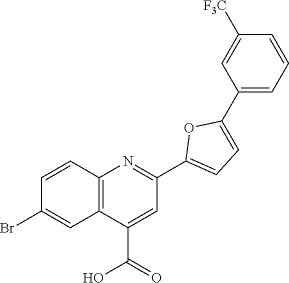 [SMILES: O=C(O)c1cc(-c2ccc(-c3cccc(C(F)(F)F)c3)o2)nc2ccc(Br)cc12] | DES-2850 |
| | 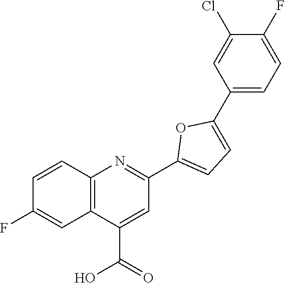 [SMILES: O=C(O)c1cc(-c2ccc(-c3ccc(F)c(Cl)c3)o2)nc2ccc(F)cc12] | DES-2851 |
| | 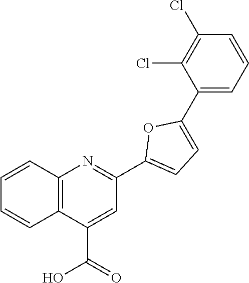 [SMILES: O=C(O)c1cc(-c2ccc(-c3cccc(Cl)c3Cl)o2)nc2ccccc12] | DES-2852 |
| | 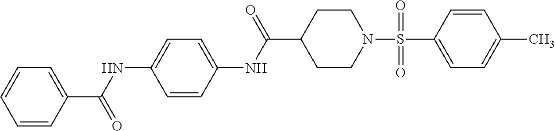 [SMILES: Cc1ccc(S(=O)(=O)N2CCC(C(=O)Nc3ccc(NC(=O)c4ccccc4)cc3)CC2)cc1] | DES-2865 |
| | 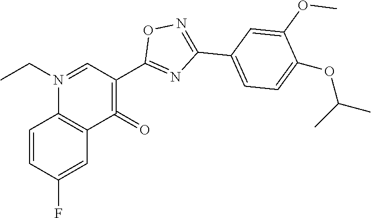 | DES-3000 |
| | 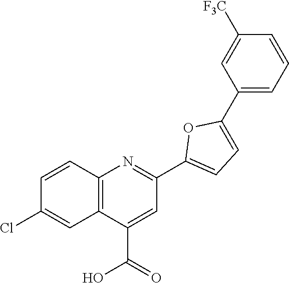 [SMILES: O=C(O)c1cc(-c2ccc(-c3cccc(C(F)(F)F)c3)o2)nc2ccc(Cl)cc12] | DES-3041 |
| 72 | 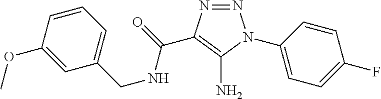 [SMILES: COc1cccc(CNC(=O)c2nnn(-c3ccc(F)cc3)c2N)c1] | DES-2089 “72” |
| | 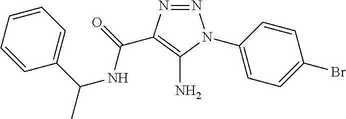 [SMILES: CC(NC(=O)c1nnn(-c2ccc(Br)cc2)c1N)c1ccccc1] | DES-2752 |
| | 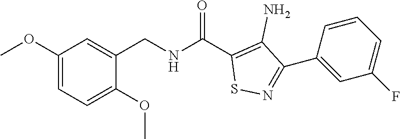 [SMILES: COc1ccc(OC)c(CNC(=O)c2snc(-c3cccc(F)c3)c2N)c1] | DES-2787 |
| | 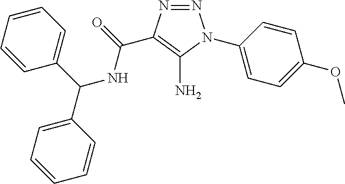 [SMILES: COc1ccc(-n2nnc(C(=O)NC(c3ccccc3)c3ccccc3)c2N)cc1] | DES-2788 |
| | 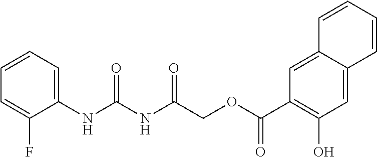 [SMILES: O=C(COC(=O)c1cc2ccccc2cc1O)NC(=O)Nc1ccccc1F] | DES-2937 |
| 91 | 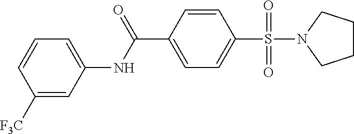 [SMILES: O=C(Nc1cccc(C(F)(F)F)c1)c1ccc(S(=O)(=O)N2CCCC2)cc1] | DES-2108 “91” |
| | 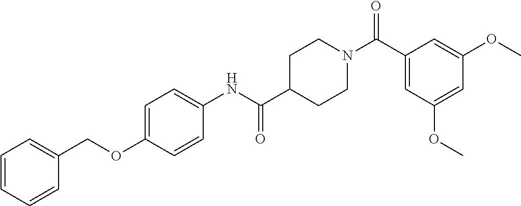 [SMILES: COc1cc(OC)cc(C(=O)N2CCC(C(=O)Nc3ccc(OCc4ccccc4)cc3)CC2)c1] | DES-2873 |
| | 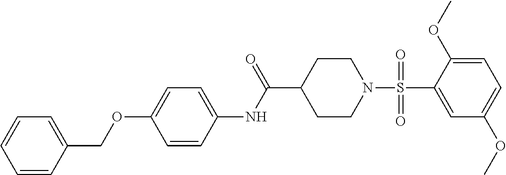 [SMILES: COc1ccc(OC)c(S(=O)(=O)N2CCC(C(=O)Nc3ccc(OCc4ccccc4)cc3)CC2)c1] | DES-2879 |
| | 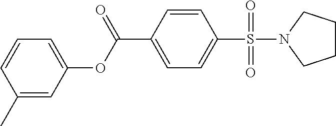 [SMILES: Cc1cccc(OC(=O)c2ccc(S(=O)(=O)N3CCCC3)cc2)c1] | DES-2900 |
| | 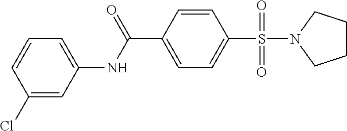 [SMILES: O=C(Nc1cccc(Cl)c1)c1ccc(S(=O)(=O)N2CCCC2)cc1] | DES-2928 |
| | 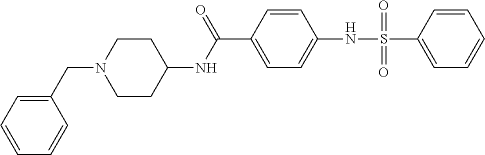 [SMILES: O=C(NC1CCN(Cc2ccccc2)CC1)c1ccc(NS(=O)(=O)c2ccccc2)cc1] | 43870447 |

A patient in need of treatment likely will be administered between 0.001 mg/kg to 15 mg/kg body weight, in particular from 0.01 mg/kg to 2.50 mg/kg body weight, in particular, from 0.01 to 1.5 mg/kg body weight, in particular from 0.1 mg/kg to 0.50 mg/kg body weight. The amount of a compound according to the present invention, also referred to here as the active ingredient, which is required to achieve a therapeutic effect may vary on case-by-case basis, vary with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to admission. As described herein below, suitable pharmaceutical formulations are prepared by known procedures using well known and readily available ingredients.

### Pharmaceutical Compositions

This invention also provides a pharmaceutical composition comprising at least one of the compounds as described herein or a pharmaceutically-acceptable salt thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying butortions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples are embedding compositions, which can be used include polymeric substances and waxes. The active ingredient can also be in micro-encapsulated form, if apbutriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the buter medium. Absorption enhancers can also be used to increase the flux of the pharmaceutical agents of the invention across the skin. The rate of such flux can be controlled, by either providing a rate controlling membrane or dispersing the compound in a polymer matrix or gel.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Generally, an effective amount of dosage of active compound will be in the range of from about 0.01 to about 1500, depending on the mode of administration. The amount administered will also likely depend on such variables as the condition to be treated, the severity of the condition, the age and overall health status of the patient, the relative biological efficacy of the compound delivered, the formulation of the compound, the presence and types of excipients in the formulation, and the route of administration. Also, it is to be understood that the initial dosage administered can be increased beyond the above upper level in order to rapidly achieve the desired tissue level or blood level, or the initial dosage can be smaller than the optimum.

Nonlimiting doses of active compound comprise from about 0.1 to about 1500 mg per dose. Nonlimiting examples of doses, which can be formulated as a unit dose for convenient administration to a patient include: about 0.10 mg, about 0.15 mg, about 0.20 mg, about 0.25 mg, about 0.30 mg, about 0.35 mg, about 0.40 mg, about 0.45 mg, about 0.50 mg, about 0.75 mg, about 1 mg, about 2 mg, about 2.5 mg, about 3 mg, about 4 mg, about 5 mg, about 7.5 mg, about 10 mg, about 12.5 mg, about 15, mg, about 20 mg, about 25 mg, about 30 mg, about 35 mg, about 40 mg, about 45 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 275 mg, about 300 mg, about 325, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, and about 1500 mg. The foregoing doses are useful for administering the compounds of the present invention according to the methods of the present invention.

Alternatively, the amount of active ingredient in the compositions useful in the methods of the present invention can be described on a weight percentage basis. Nonlimiting amounts of active ingredients include about 0.01%, about 0.015%, about 0.02%, about 0.025% about 0.03%, about 0.035% about 0.04%, about 0.045%, about 0.05%, about 0.055%, about 0.06%, about 0.065%, about 0.07%, about 0.075%, about 0.080%, about 0.085%, about 0.090%, about 0.095%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.25%, about 1.5%, about 1.75%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, and about 99.9%.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another compound for treating neurodegenerative diseases), or they may achieve different effects (e.g., control of any adverse effects).

The compounds of the invention may be administered intravenously, intramuscularly, intraperitoneally, subcutaneously, topically, orally, or by other acceptable means. The compounds may be used to treat conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name.

### Example 1: Determining α-Synuclein Toxicity Rescue in Yeast

Yeast Strains and culturing: Yeast strains expressing alpha-synuclein have been described in Cooper at. al, 2006. (Cooper A A, et al. Alpha-synuclein blocks ER-Golgi traffic and Rab1 rescues neuron loss in Parkinson's models. Science. 2006 Jul. 21; 313:324) Strains express multiple copies of alpha-synculein for galactose-inducible expression. In addition, all stains have either deletions of the Δpdr1::KanMX and Δpdr3::KanMX or Δpdr5::KanMX to reduce efflux of compounds and reduce the required dose of compounds. Yeast were cultured in complete synthetic media (CSM) and an appropriate dropout (lacking histidine or uracil) to maintain plasmids if required. For galactose-induction experiments, overnight cultures were grown in CSM/2% glucose to saturation and diluted 1:20 into CSM/2% raffinose for ˜2 generations. Cultures were then diluted into CSM/2% galactose at an optimum OD₆₀₀ for the experiment (see ‘Growth assays’).

Deletion strains were generated by transforming WT yeast with a PCR product of the HygromycinR cassette with 5′ and 3′ flanking sequences of the gene to be deleted. PCR products were purified (Qiagen, MinElute), verified by agarose gel electrophoresis, and transformed into competent yeast using LiOAc-based transformation. Cells were grown in rich media (YPD) for ˜4 hrs before plating on YPD/Hygromycin plates. Genetic disruption was confirmed by PCR using oligonucleotides upstream of the deletion and a reverse oligo within the HygR gene. For deletions in the α-syn-expressing yeast, deletions were generated in opposite mating type and mated, sporulated, and dissected to obtain the correct genotypes. Correct markers and mating type were confirmed.

GFP-tagged strains (MUP1-GFP and SNA3-GFP) were generated by homologous recombination of a PCR product amplified from the GFP-tagged library in yeast strain BY4741 (Open Biosystems). Transformants were selected on SDHis plates and correct integration confirmed by PCR, fluorescence microscopy, and western blotting.

WT or α-syn strains harboring plasmids were constructed by LiOAc transformation of empty vector (e.g., pAG413/416Gal-ccdb) or pAG413/416Gal-ORF. Transformations were plated on synthetic drop-out lacking either histidine or uracil for selection of the plasmid. All subsequent husbandry used appropriate drop-out media.

Plasmids: Plasmid construction for galactose-inducible overexpression experiments was accomplished by transferring ORFs from the FlexGene library (30) to pDONR221 using BP Clonase (Invitrogen) according to manufacturer's specifications. Entry clones were verified by BsrGI restriction digests and, if needed, DNA sequencing. After verification, ORFs were transferred to Gateway-compatible destination vectors (pAG413Gal) using LR Clonse (Invitrogen) according to manufacturer's specifications. Clones were verified by BsrGI restriction digests. Generated plasmids are listed in Table S2.

Yeast Growth assays: Starting cultures for all dose-response assays were based on strains initially constructed in the lab to maintain homogeneity across experiments. All growth assays were carried out in 384 well format. Source plates were assembled in 96 well plates using multichannel pipettes to dilute rows in 1.6-fold serial dilutions of CSMGal. To these dilution series containing 2× final concentration of compound, 2×OD₆₀₀ culture (in CSMGal) was dispensed with a multichannel pipette to achieve a final drug/culture mix with the desired OD₆₀₀ and drug concentration. For WT yeast, the final starting OD₆₀₀ was 0.01. For α-syn, the final starting OD₆₀₀ was 0.02. Drug concentration ranges varied depending on efficacy, growth inhibition, and solubility in media. A Tecan EvoFreedom liquid handling robot was then used to transfer culture from 96 to 384 well format with each well being represented four times. Final well volume was 35 μL. Plates were then incubated in humidified containers at 30° C. for either 24 or 40 hours. Plates were then read with a Tecan Saphire plate reader at OD₆₀₀.

Raw OD₆₀₀ values were transformed to “Relative Growth” in WT cells or “% Maximum Rescue” in α-syn experiments. In WT cells, the well background was subtracted and all values were then normalized to 100% for the untreated condition. In α-syn rescue experiments, the well background was subtracted and the maximum rescue in the particular experiment was normalized to 100%. All experimental data points were then calculated by (OD_{600Exp}−OD_{600untreated}/(OD_{600Max}−OD_{600untreated})×100 to obtain rescue relative to maximum rescue observed. Dose-response curves were generated by nonlinear regression analysis using Prism Graphpad v. 6.0. In cases where the compounds began inhibiting growth, only points up to the maximum were used to fit the curve. Above that, points were directly connected and are always presented as dotted lines.

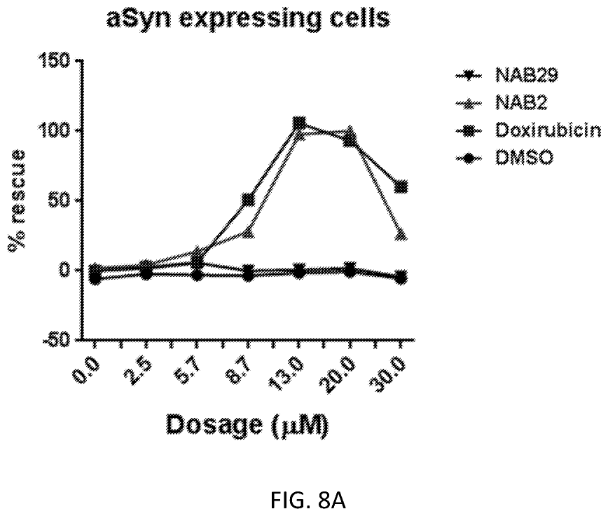

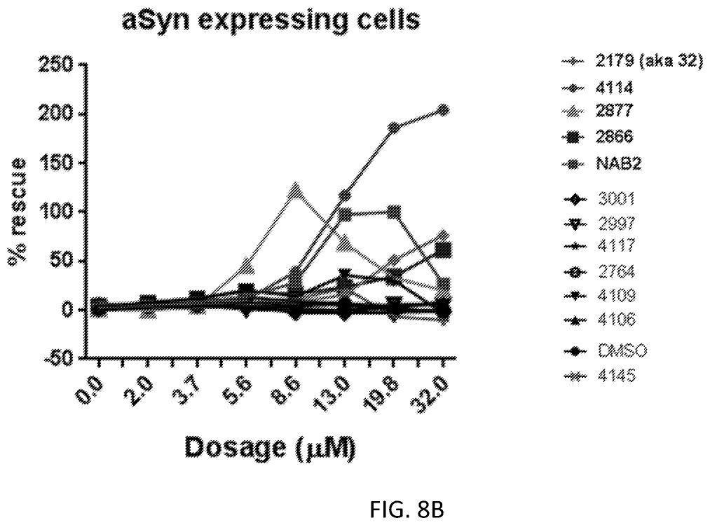

The effect of compounds on rescue of aSyn toxicity in yeast are shown in FIGS. 8A-8B. DES-2877 and DES-4144 were most effective in rescuing aSyn toxicity in yeast. DES-2866 and DES-2184 were also effective in rescuing aSyn toxicity in yeast.

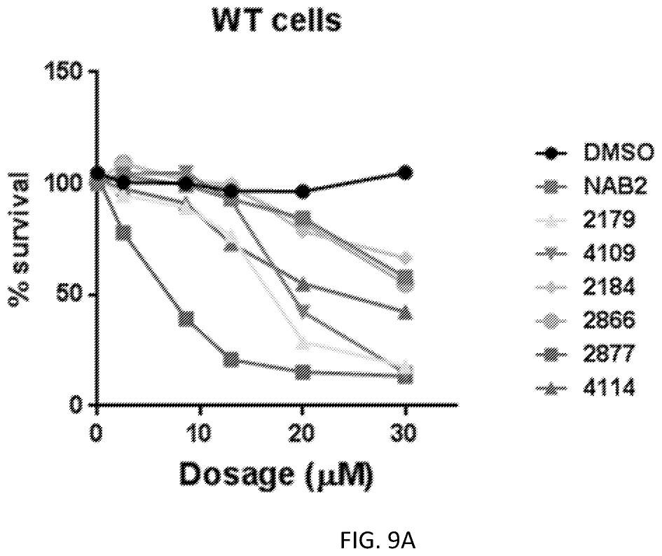

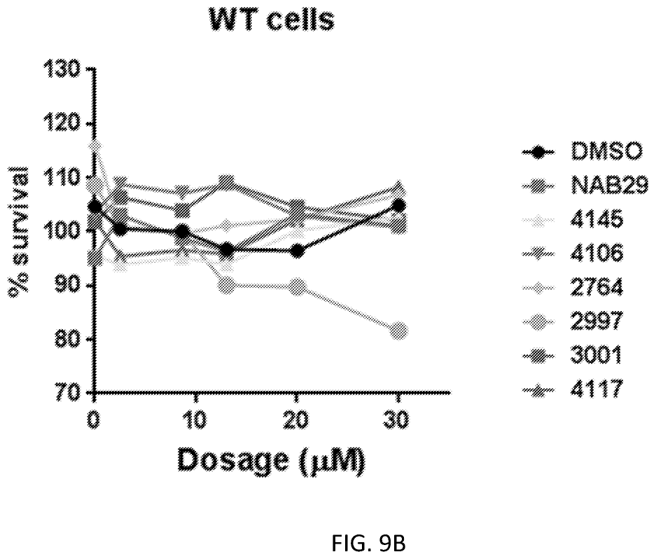

The toxicity profiles of compounds on WT control yeast strain are shown in FIGS. 9A-9B. Compounds that were active in rescuing synuclein all showed toxicity to some extent. DES-4114 was the least toxic among active analogs, and also the most effective in rescuing aSyn toxicity. Inactive compounds were not toxic in WT yeast cells.

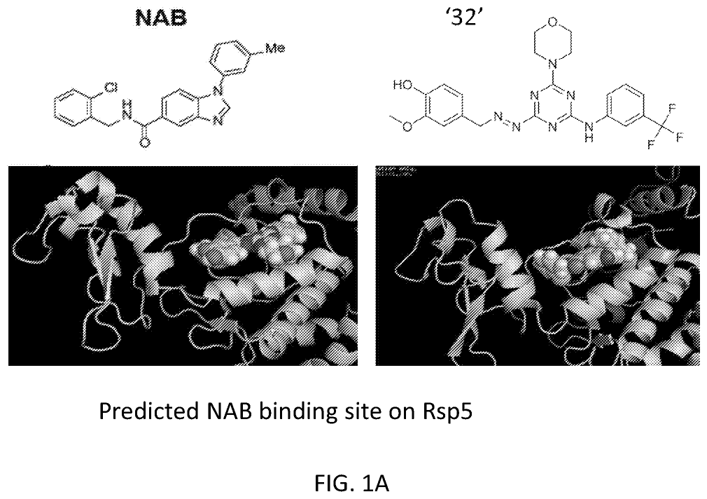

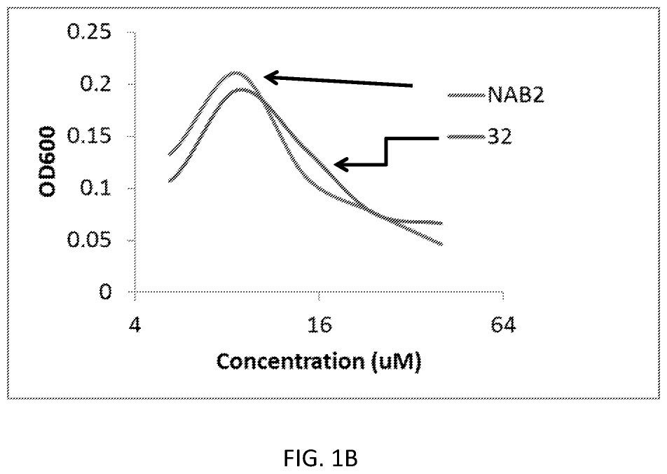

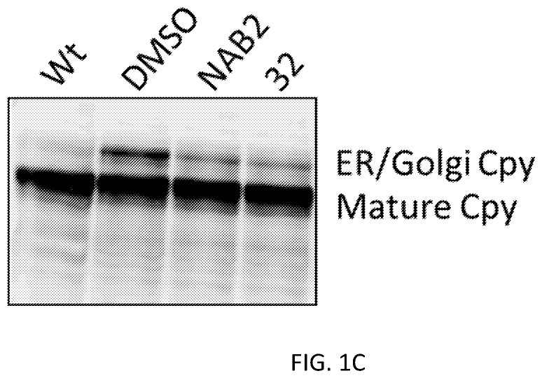

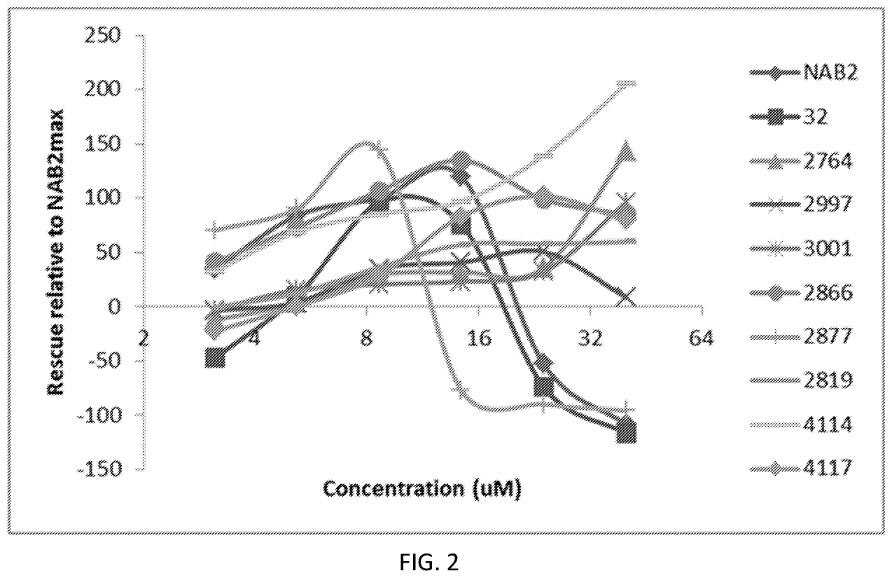

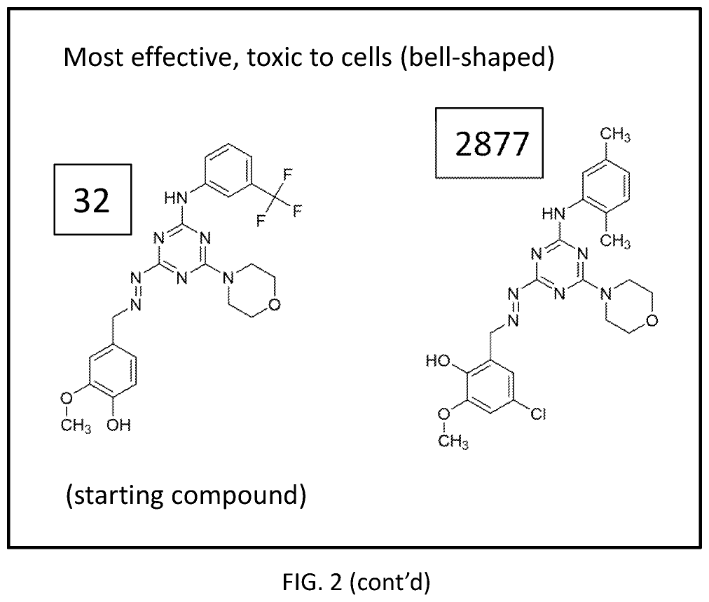

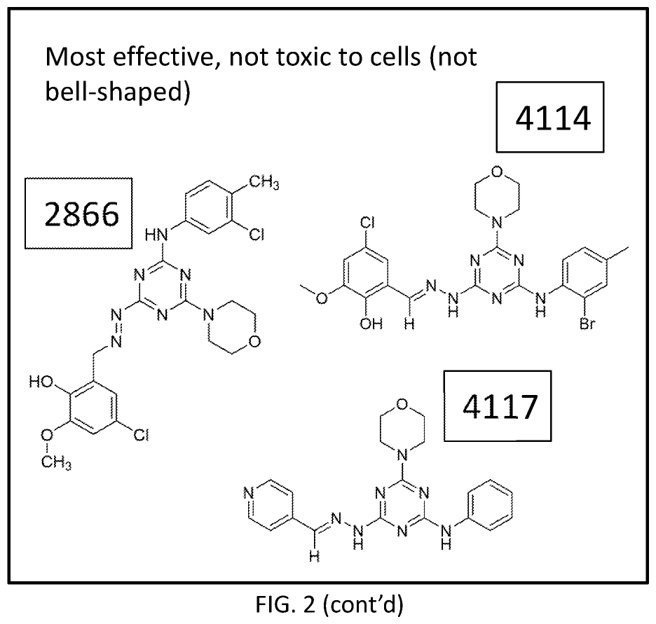

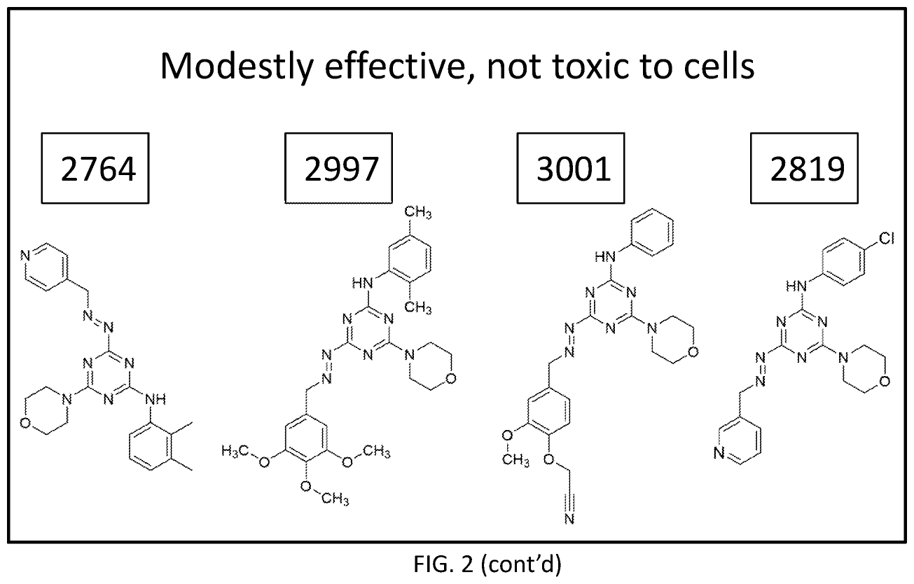

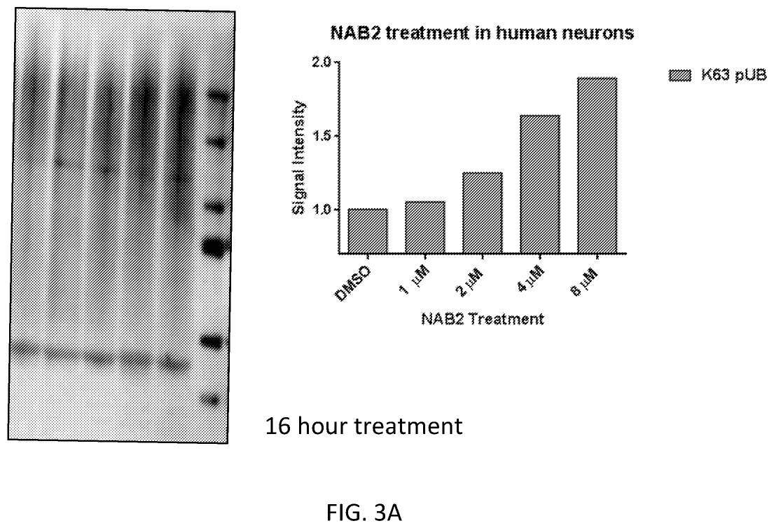

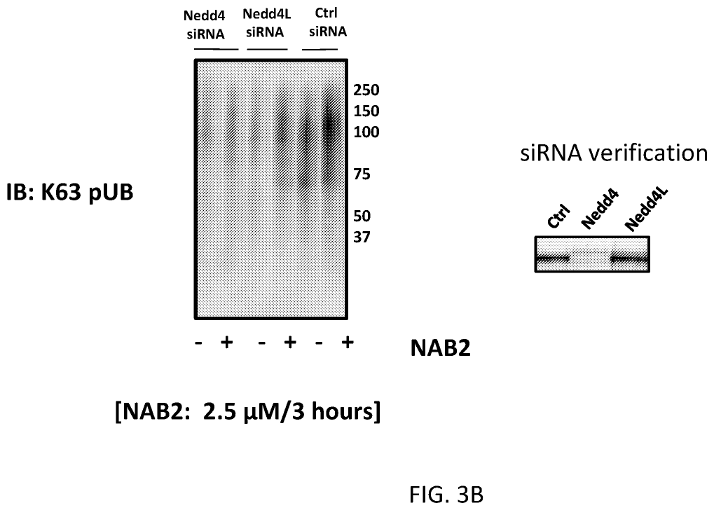

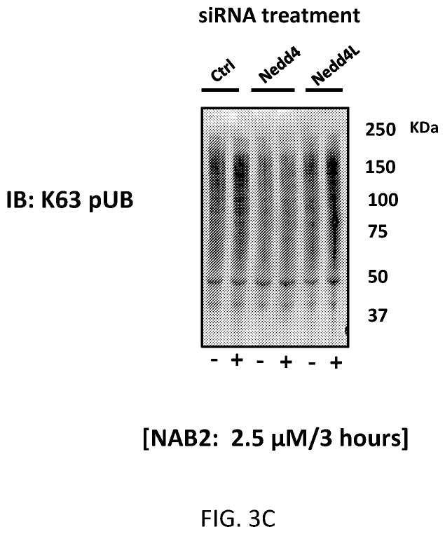

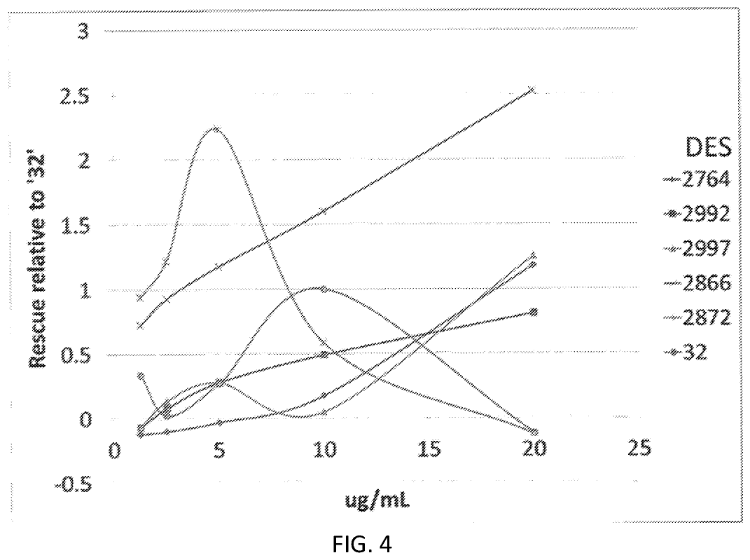

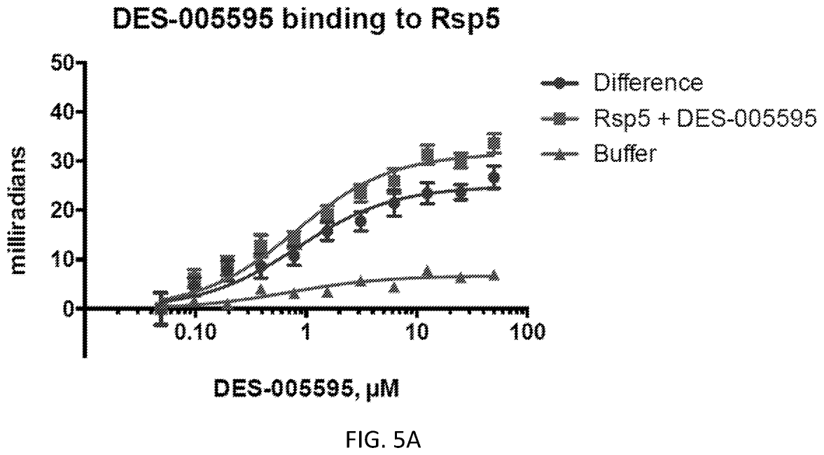

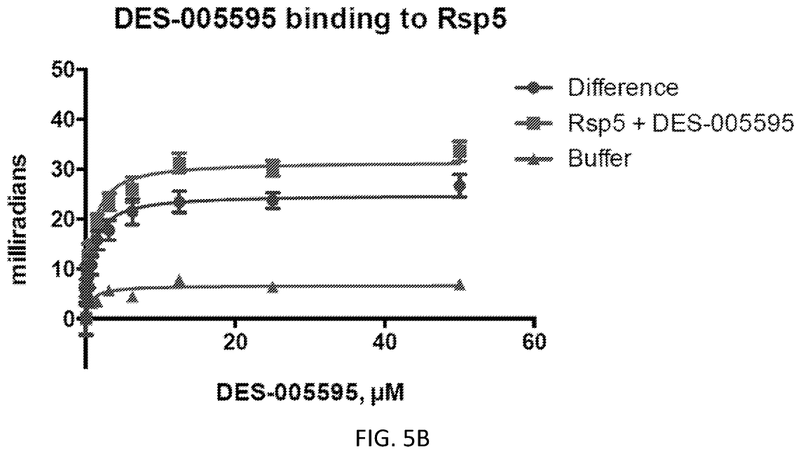

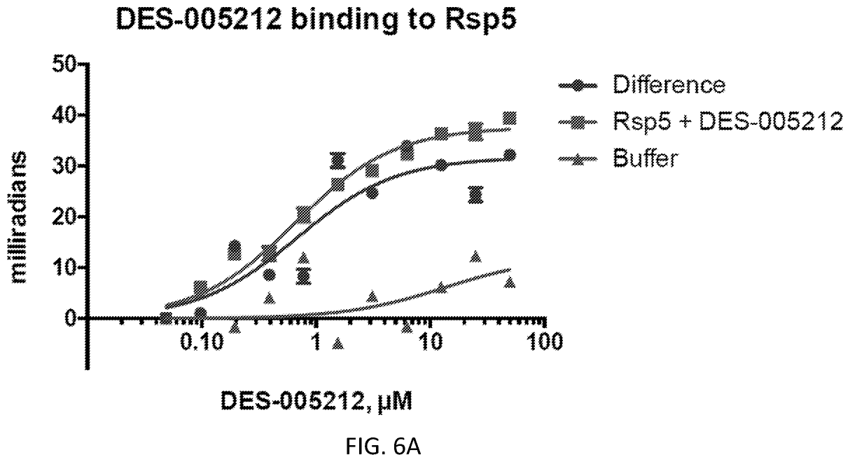

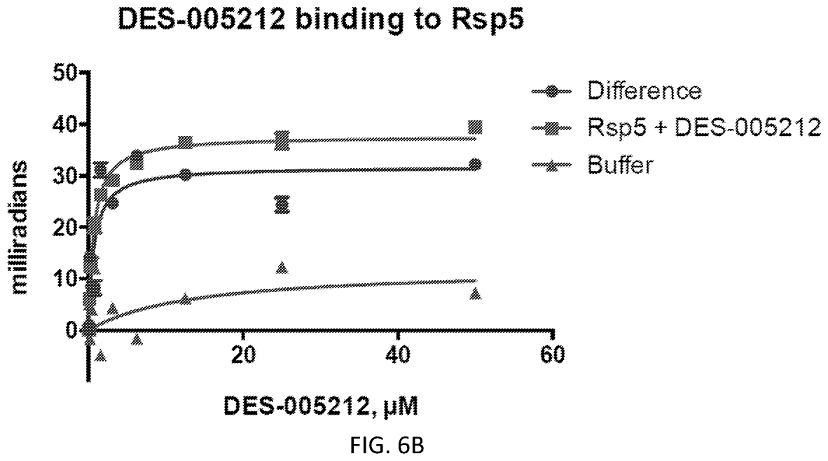

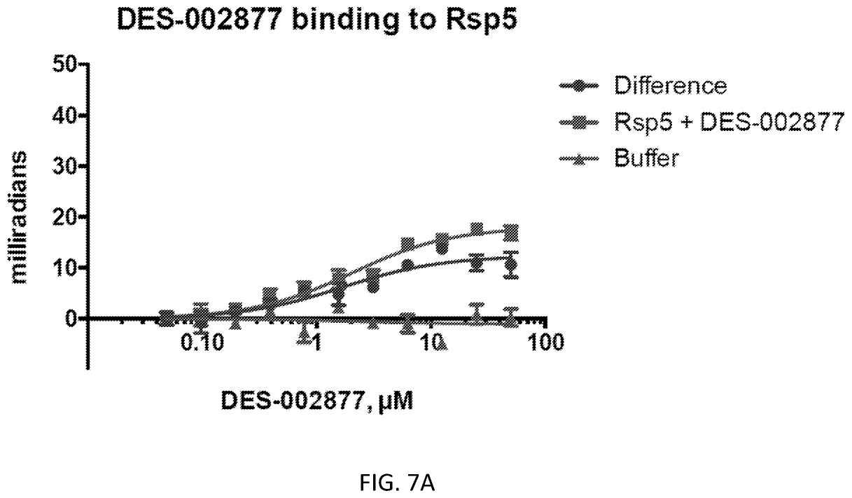

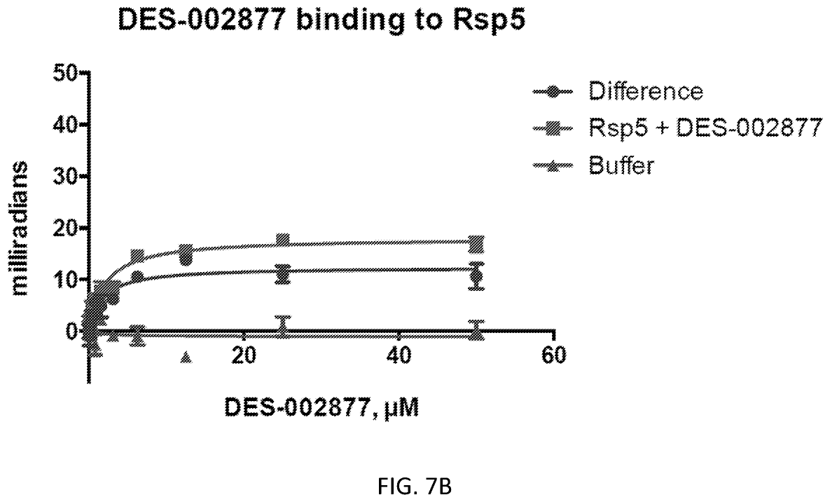

Representative dose-response curves of sample compounds that show some activity in rescuing α-synuclein toxicity in yeast are shown in FIG. 2. Dose-response curves are also shown in FIG. 1B, wherein α-synuclein-expressing yeast was treated with increasing concentrations of both NAB2 and ‘32’. Efficacy increases to a peak around 10 μM and then NAB2/′32′ begin to slow growth, most likely due to over activation of Rsp5.

### Example 2: Immunoblot Analysis of Sna3-GFP Polyubiquination and Cpy Trafficking Intermediates Enroute to Vacuole

Protein analysis was performed in NoTox and HiTox strains with the compound treatment at indicated concentrations. Log phase CSM/2% raffinose cultures were induced with 2% galactose for 5 hours with DMSO or the compounds. Cultures were normalized to cell density and cell pellets prepared for SDS-PAGE. Cell pellets were boiled in SDS-loading dye for 15′, centrifuged, and resolved by 4-12% SDS-PAGE. CPY western blots were performed using culture conditions as described above. An anti-Cpy antibody (Invitrogen, A6428) was used at 1:5,000. Post-ER:ER ratios were quantitated using an IRDye800 secondary antibody (Li-Cor Odyssey, Rockland Immunochemicals) and scanned with the Li-Cor Odyssey imaging system. Significance was determined using a one-way ANOVA and Tukey's test of significance. From the same gel, total protein was detected by coomassie staining. Both blots and coomassie-stained gels were scanned using the Li-Cor Odyssey imaging system and quantitated. Significance was determined using a one-way ANOVA with Tukey's test of significance.

In WT Sna3-GFP cells, log phase CSMRaf cultures were shifted to galactose for 5 hours in the presence or absence of the compounds. Cell pellets were lysed in SDS-loading dye and Sna3-GFP cleavage monitored by Western blotting with an anti-GFP antibody. For Sna3-GFP analysis in α-syn cells, a strain in which GFP was integrated at the chromosomal locus of SNA3 in our untagged α-syn strain was used. Log phase CsmRaf cultures of WT or α-syn yeast were shifted to galactose for 5 hours in the presence or absence of the compounds at which point they were then prepared for Western blot analysis.

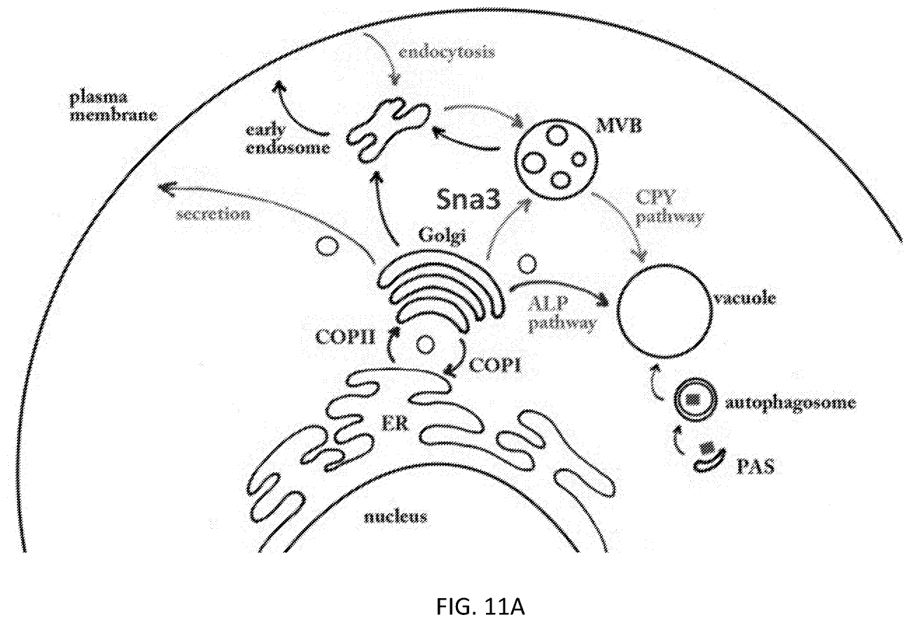

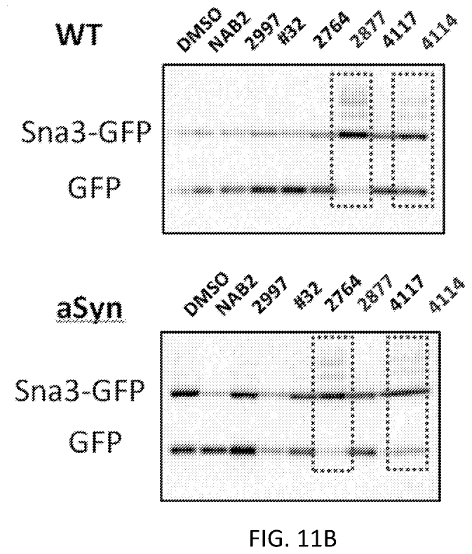

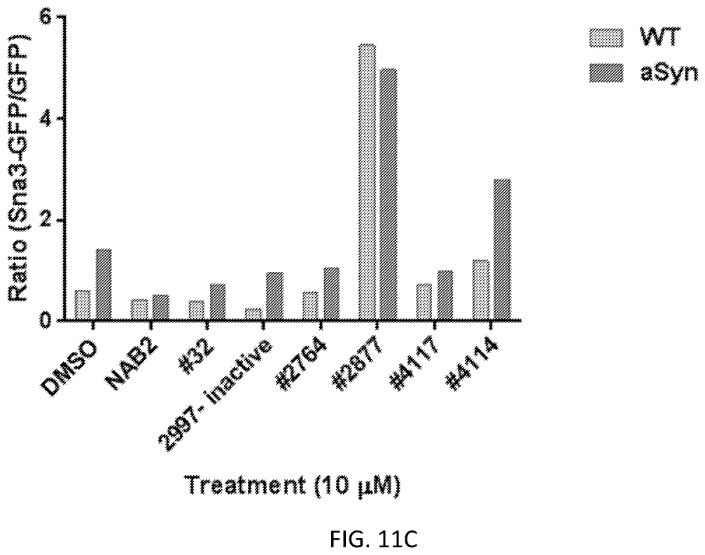

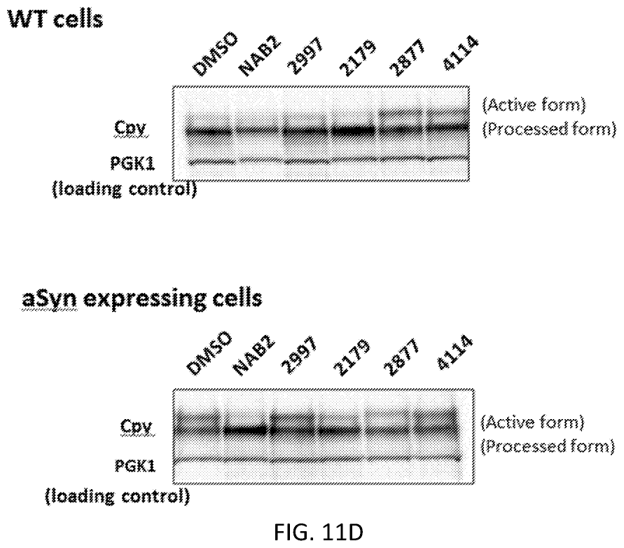

The effect of sample compounds on ubiquitination of Sna3-GFP in WT and α-syn cells is shown in FIG. 11B. DES-2877 and DES-4114 cause an increase in the polyubiquitinated Sna3-GFP. The ratio of Sna3-GFP to free GFP for these compounds in WT and α-syn cells is shown in FIG. 11C. The effect of compounds on Carboxypeptidase Y (CPY) trafficking intermediates enroute to the vacuole is shown in FIG. 11D. DES-2877 and DES-4114 cause an increase in accumulation of CPY trafficking intermediates en route to the vacuole.

As shown in FIG. 1C, Western blot analysis of Cpy shows that Cpy is differentially cleaved when trafficking from the Endoplasmic Reticulum to the Golgi and Vacuole. Accumulation of the high molecular weight band reflects a block in vesicle trafficking. Both NAB and ‘32’ ameliorate this defect.

### Example 3: Morphological Analysis aSyn-Expressing Yeast Cells

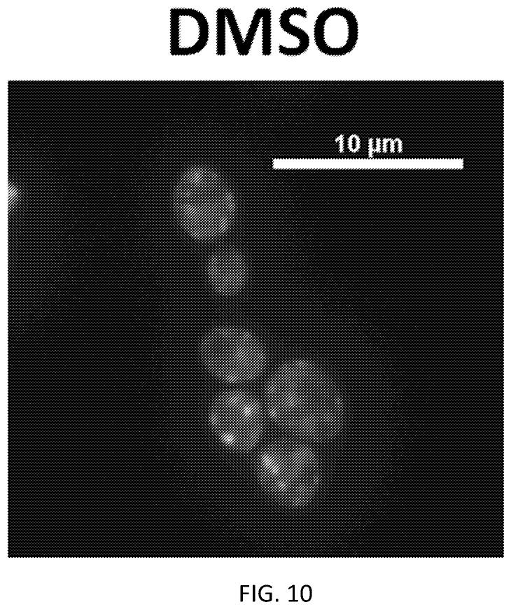

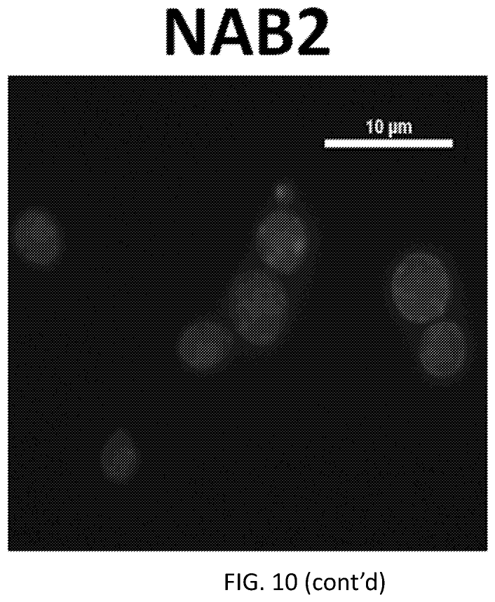

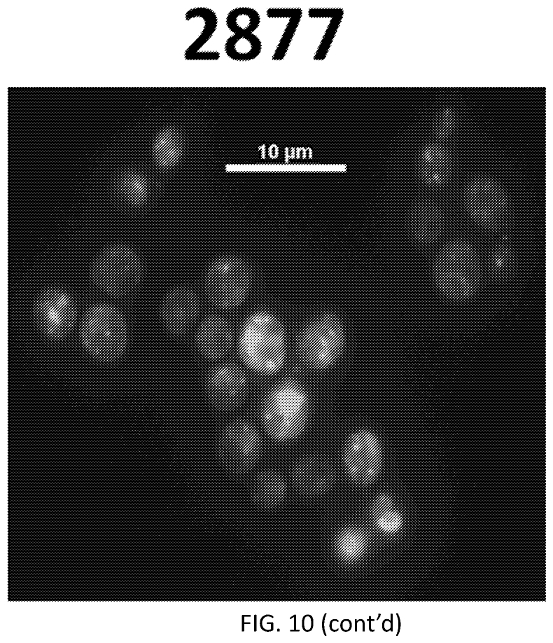

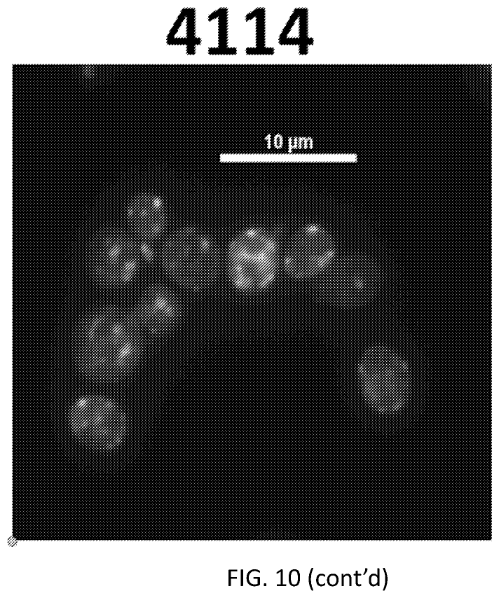

Morphological analysis shows that rescue of aSyn toxicity by DES-2877 and DES-4114 is accompanied by an accumulation of vesicular intermediates in yeast cells. Raffinose cultures of α-syn expressing yeast cells were grown up to the logarithmic phase in raffinose. Cultures were induced with galactose for five hours in the presence or absence of the indication concentration of the compounds. In the present example, the identified compounds were present at a concentration of 10 uM. Cells were centrifuged, media discarded, and then fixed with 4% paraformaldehyde in 1×PBS for 1 hr. The fixed culture was centrifuged, and the pellet resuspended in 0.4% paraformaldehyde in 1×PBS and kept at 4° C. Single plain images were taken at 100× magnification with a Nikon Eclipse Ti microscope and are provided in FIG. 10.

### Example 4: Binding to the HECT Domain of Recombinant Rsp5

Back-Scattering Interferometry (BSI) is a label-free, free-solution technology that employs novel, conformation-sensitive detection to characterize complex drug targets-small molecule interactions in a native-like environment. (For a review of Back-Scattering Interferometry, see, e.g., D. J. Bornhop et al., Science 2007, 317 (5845), 1732-6; and references cited therein; each of which hereby incorporated by reference in its entirety.) Back-Scattering Interferometry can be used, e.g., to detect of specificity conformational change, engage target molecules, and/or detect allosteric modulation. Exemplary advantages of back-scattering interferometry include target-ligand binding specificity for complex targets and matrices; radio-assay like sensitivity in a label-free, in-solution, tether-free assay format; mass-independent sensitivity in complex matrices to enable small molecule-large target studies; direct K_{d} determination for both inactive and active enzymes; and affinity vs efficacy based allostery.

Rsp5 is an E3 ubiquitin ligase that transfers ubiquitin from an E2 ubiquitin-conjugating enzyme to its specific substrate for degradation at the proteasome. The HECT domain of Rsp5 contains an N-lobe for E2 binding and a C-lobe for ubiquitin transfer. Rsp5 is involved in the endocytosis of plasma membranes permeases, the biosynthesis of unsaturated fatty acids and heat-shock element mediated gene expression.

Sample Preparation of Rsp5 Target: Rsp5 was supplied in 15 μL aliquots of 100 μM (25 mM HEPES pH 7.5, 200 mM NaCl, 5 mM DTT) by St. Jude Research Hospital (Memphis, Tenn.) and was stored at −80° C. Immediately prior to assays fresh aliquots were thawed and diluted in 25 mM HEPES, pH 7.5, 200 mM NaCl, 1 mM DTT, 0.005% pluronic acid and 1% DMSO. The final Rsp5 concentration in the binding assay was 100 nM.

Sample Preparation of compounds (ligands): The compounds were received as solids and reconstituted to either 40 or 20 mM in 100% DMSO and stored at −80° C. in single use aliquots. The final concentration of each ligand in an assay was 50 μM with a 2× serial dilution to create a 12-point curve.

The assay buffer was 25 mM HEPES, pH 7.5, 200 mM NaCl, 1 mM DTT, 0.005% pluronic acid, 1% DMSO. The assay was run in Eppendorf 96-well PCR microplates. 55 μL of either Rsp5 or buffer (as control) were added to a each well. To these wells were added 55 μL of the compound dilution. A reference channel containing only buffer was setup as well for thermal compensation during assay measurements. The plates were heat sealed with foil and the assay plates were allowed to incubate at room temperature for 2 hours. Wells were pierced individually prior to sample injection and measurement of BSI signal (each well analyzed in duplicate). The assays were run using a glass microfluidic chip with a proprietary surface treatment on TruBind™ 100 system.

The BSI signal was expressed as the magnitude of the spatial shift of the fringe pattern on a CMOS camera, measured in milliradians. The control signal was subtracted from assay signal for each compound dilution point. The resulting values were re-zeroed and analyzed with the GraphPad Prism program. The dissociation constant (K_{d}) was derived from non-linear least-squares fitting of the data using the one-site saturation binding model. The goodness of fit was judged by the calculated R² value. The difference and control curves for at least two successful assays were averaged. The resulting average difference curve was used to calculate the reported K_{d} value for each compound.

NAB2-01, DES-002877-04, and DES-005212-01 demonstrated low- to sub-μM binding to Rsp5, with dissociation constants of 0.84±0.12 1.7±0.4 and 0.68±0.18 respectively. Three compounds, DES-5596, DES-4117, DES-3001, did not show binding to the target. These compounds either saw high ligand control BSI signal (DES-5596 & DES-4117) or minimal assay response (DES-3001) in general.

### Example 5: Determining α-Synuclein Toxicity in Yeast Primary Rat Neuronal Culture

Cultures were prepared based on Lesuisse and Martin (Lesuisse et al., Journal of neurobiology 51.1 (2002): 9-23; hereby incorporated by reference in its entirety). Embryos were harvested by Cesarean section from anesthetized pregnant Sprague-Dawley rats at embryonic day 18. Cerebral cortices were isolated and dissociated with ACCUMAX digestion for 20 min at 37° C. and trituration with Pasteur pipettes. Polyornithine and laminin-coated 96-well plates were seeded with 4×104 cells in neurobasal medium (Life Technologies) supplemented with B27 (Life Technologies), 0.5 mM glutamine, 25 μM β-mercaptoethanol, penicillin (100 IU/mL), and streptomycin (100 μg/mL). One third of the medium was changed every 3-4 days. Compounds were added at the indicated concentrations to the cultures in 96-well plates at day in vitro (DIV)18 keeping the amount of DMSO constant (vehicle). As a surrogate marker of cell viability, cellular ATP content was measured using the ViaLight Plus kit (Lonza).

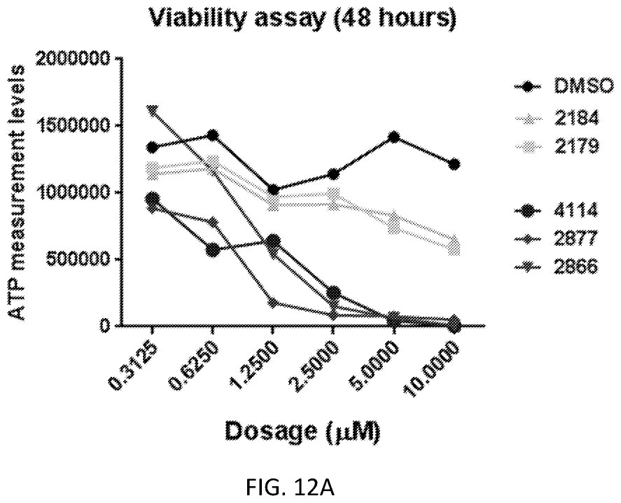

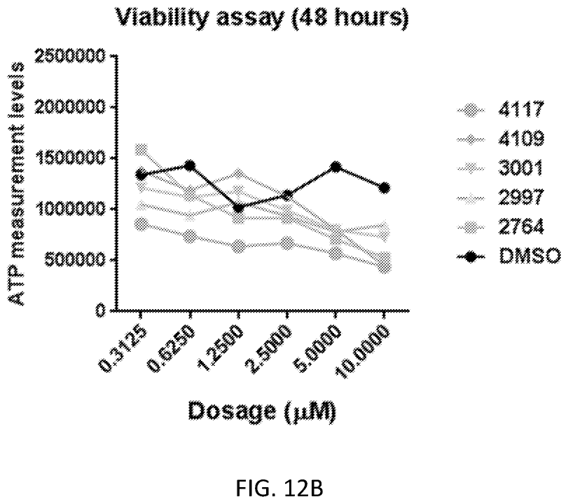

The toxicity profiles of compounds on rat cortical neurons are shown in FIGS. 12A-12B (for DES-2184, DES-2179, DES-4114, DES-2877, DES-2866, DES-4117, DES-4109, DES-3001, DES-2997, and DES-2764). The compounds that were active in rescuing aSyn were toxic in rat cortical neurons. The less effective compounds were less toxic. 24 hour time point showed identical trends.

### Example 6: Effect on K63-Ub in Human Cells

### Generation of iN Neurons.

iN neurons were made from an inducible NGN2 hPSC line based on the findings from Zhang et. al, 2013. Briefly, hPSCs were dissociated with Accutase and plated at a density of 750000 cells in a 6 well plate with 2 mls of 1:1 mTest:MEF conditioned media with Rock inhibitor. Cells were transduced with NGN2:Puro lentivirus and UbC-rtTA virus and incubated for 24 hours. Media with virus was replaced with 1:1 mTesr:MEF media with 10 μg/ml Rock inhibitor. After 24 hours, mTesr:MEF media was replaced with mTesr media and passaged five times, before beginning differentiations. For differentiation, Dox-NGN2 inducible stem cells line were plated at 750,000 cells per well of a Matrigel coated 6-well plate in the presence of mTesr with 10 ug/ml Rock inhibitor and 2 ug/ml doxycycline. After 24 hours, mTesr media was replaced with Neurobasal N2/B27 media with Puromycin and doxycycline. On day 7 neurons were replated at the required density in the presence of Neurobasal N2/B27 media without Doxycycline with neurotrophic factors [BDNF: 10 ng/ml, GDNF: 10 ng/ml, cAMP: 1 mM, Ascorbic Acid: 0.2 μM; Laminin: 1 μg/ml] and AraC [0.5 μM] to eliminate glia. On day 11, media was changed to 1:1 Neurobasal and BrainPhys media with N2/B27. From day 14, N2/B27 BrainPhys media supplemented with neurotrophic factors was used to maintain the differentiated neurons.
1×N2 (Gibco, Cat No. 17502-048)
1×B27 (Gibco, Cat No. 17504-044)
Brain-derived Neurotrophic Factor (BDNF, 40 ng/ml; Peprotech, Cat No. 450-02)
Glia-derived Neurotrophic Factors (GDNF, 40 ng/ml; Peprotech, Cat No. 450-10)
ascorbic acid (AA, 400 nM; Sigma, Cat No. A0278)
dibutyryl cyclic AMP (cAMP, 2 mM Sigma, Cat No. D0627)
laminin (2 μg/ml; Invitrogen, Cat No 23017-015)
10% FBS (Gibco, Cat No. 10082-147)
0.5 uM AraC
   p-Ub Chain Linkage Pull-Down Protocol

Human HEK-293 or neuronal cells treated with the appropriate compounds, were washed twice with ice cold PBS and then 0.5 ml of Lysis buffer was added and cells were scraped off the 10 cm dish (1 ml for 15 cm).

Lysis buffer is 50 mM Tris/HCl pH 7.5, 1 mM EGTA, 1 mM EDTA, 0.5 or 1% (v/v) NP-40, 1 mM sodium orthovanadate, 50 mM NaF, 5 mM sodium pyrophosphate, 0.27 M sucrose, 10 mM sodium 2-glycerophosphate, 0.2 mM phenylmethylsulphonyl fluoride, 1 mM benzamidine, plus 100 mM iodoacetamide added fresh prior to lysis (weight powder, don't use a frozen stock solution) to inactivate deubiquitylase activities and add also pepstatin/aprotinin to inhibit proteases. Cell extracts were sonicated twice for 15 seconds each time and clarified by centrifugation at 14000 g for 15 min at 4° C. Supernatants were collected and filtered using a 0.45 uM MiniSart/Syringe. Next, protein concentrations were determined by Bradford procedure.

The avidity based K63 linkage sensor protein was based on Sims et. al, 2012. Briefly, avidity based K63 sensor Halo-fusion protein was expressed in an E. coli expression vector and covalently bound to Halo-tag beads [Magne® HaloTag® Beads, 20% Slurry; Cat #G7281]. To capture poly-ubiquitylated proteins, 1 mg of cell extract protein was incubated for 3 h to 0/N at 4° C. with affinity resin bound to K63 linkage based avidity sensor. After incubation, the beads were washed three times with 1 ml of Lysis buffer containing 500 mM NaCl and once with 0.5 ml of 10 mM Tris/HCl pH 8.0. The beads are then transferred to a Spin-X centrifuge Tube filters and spun down twice for 1 minute at 2000 g and flow through discarded. The captured proteins are released by adding 1× Laemelli Sample Buffer (40 ul) onto the beads and after a quick vortex, the beads are removed by centrifuging the Spin-X tube for 2 minutes at 6000 g and flow through collected. The eluate is heated at 75° C. for 5 min and analysed by immuno-blotting using an anti-K63 linkage specific antibody (http://www.abcam.com/ubiquitin-linkage-specific-k63-epr8590-448-antibody-ab179434.html).

### Random Mutagenesis of Rsp5

Random mutagenesis of Rsp5 was performed on the HECT domain between the amino acids 565-809. pAG414-Rsp5 was cut with NsiI/BtgI and the fragment was gel purified. On the resulting fragment as template, PCR was performed with the GeneMorph II random mutagenesis kit using the following primers.

Fwd primer:
(SEQ ID NO: 1)
TGTGGGTCTTGGTGTTTTCCATAGAAGATTTTTGGATGCATTCTTTGTAG
GTG
Rev primer:
(SEQ ID NO: 2)
TGCGGAATAATCATTCTTGACCAAACCCTATGGTTTCTTCCACGGCCAAT
GTTAGCT

The resulting PCR products were purified and ligated back into the vector using Gibson Assembly and transformed into yeast. Mutants resistant to the compound treatment were selected by dispensing the library of Rsp5 variants in 384 well plates at an OD600 of 0.01. Drug resistant clones that grew out after 3-4 days were validated and checked against other toxic compounds. Plasmid DNA from 5 mls of saturated cultures were isolated using Zymoresearch DNA isolation kits to maximize recovery. The sequence variants were amplified by PCR and sequenced using the following primers.

Fwd primer:
(SEQ ID NO: 3)
GGCGTGGTTAACGTCCGCGTGGG
Rev primer:
(SEQ ID NO: 4)
CCCTATGGTTTCTTCCACGGCC

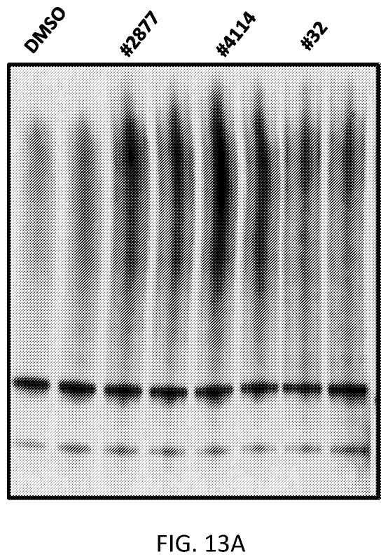

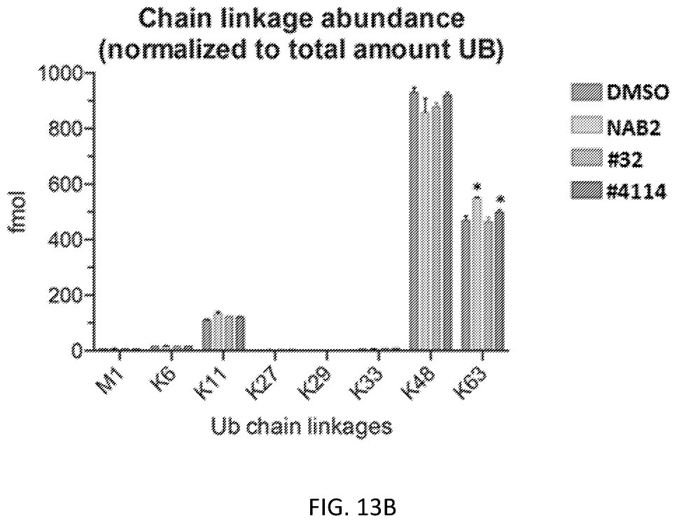

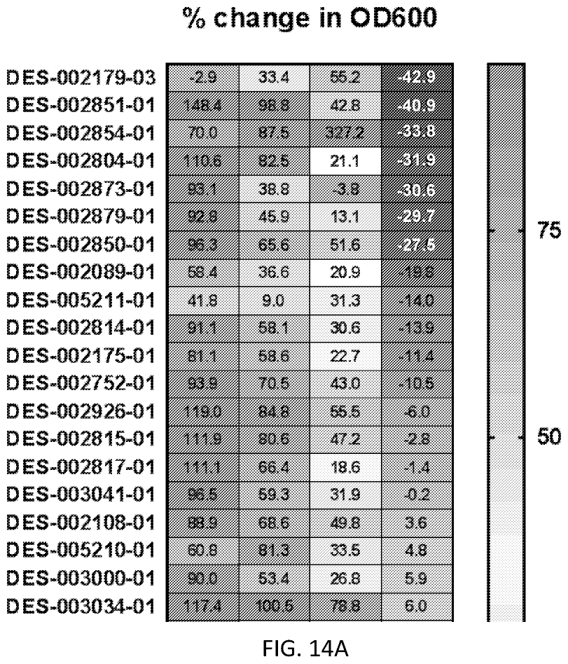

Pure neuronal cultures derived from human iPS cells were treated with DMSO, DES-2877, DES-4114 and #32 at 5 μM for 12 hours. As shown in FIG. 13A, DES-4114 and #2877 caused a modest increase in K63 linkages in iPS derived human neurons. FIG. 13B provides the results relating to HEK-293 cells treated with DMSO, NAB2, #32, and DES-4114 at 5 μM for 12 hours. For each Ub chain linkage, the order of compounds, from left to right, is DMSO, NAB2, ‘32,’ and DES-4114. DES-4114 caused a modest increase in K63 linkages in human HEK-293 cells. Poly-UB capture was performed with immobilized Halo-UBA^{UBQLN1} prior to AQUA proteomics with a library of ¹³C/¹⁵N-labeled reference peptides (Phu et al., Improved quantitative mass spectrometry methods for characterizing complex ubiquitin signals. Mol Cell Proteomics. 2011; 10 M110 003756). Ubiquitylation site identification by mass spectrometry was performed as described Kim et al., (Systematic and quantitative assessment of the ubiquitin-modified proteome. Mol Cell. 2011; 44:325-340). and Sarraf et al., (Landscape of the PARKIN-dependent ubiquitylome in response to mitochondrial depolarization. Nature. 2013; 496:372-376).

### Example 7: Screening Analogs for Ability to Rescue aSyn Toxicity for Better Physicochemical Properties

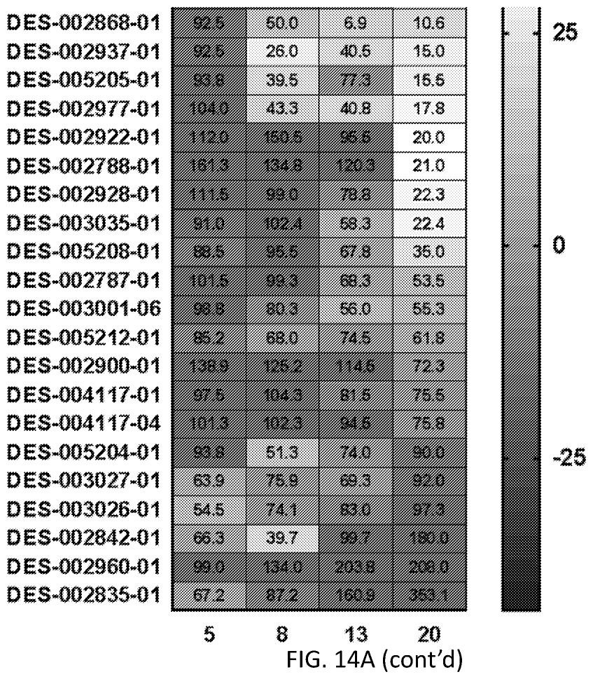

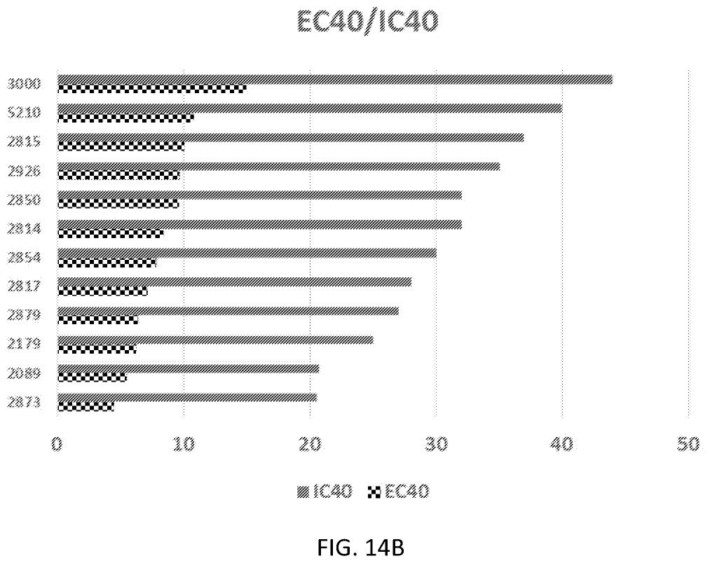

A number of analogs of six series of compounds (#32, #37, #4117, #72, #91, #28) were tested for ability to rescue alpha-synculein toxicity in yeast model. Various sample compounds were identified as positive ‘hits’ in the toxicity rescue screen and were able to rescue aSyn toxicity with various levels of efficacy. Further examination of structure activity relationships, shows a correlation between potency and growth inhibition. FIG. 14A shows a heatmap representation of aSyn toxicity rescue for selected samples. The heatmap shows the percent change in OD600 as compared to untreated yeast cells expressing alpha-synuclein. Compounds were tested at a range of different concentrations ranging from 5 uM to 20 uM to maximize the window of effective concentrations. The test samples are identified in FIG. 14A according to the naming convention described herein followed by a two digit suffix. The two digits after the compound number refer to the different batches of compounds that were obtained from different sources and tested. FIG. 14B shows the EC₄₀ and IC₄₀ values for selected compounds represented in FIG. 14A.

### Example 8: Functional Screening of Compound Hits on Ability to Promote Sna3-GFP Trafficking

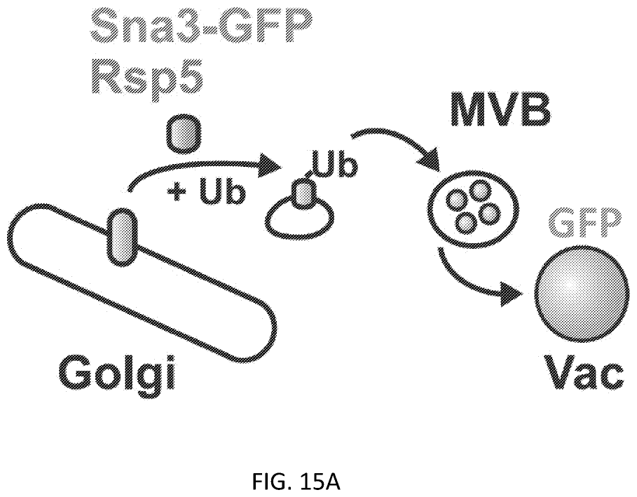

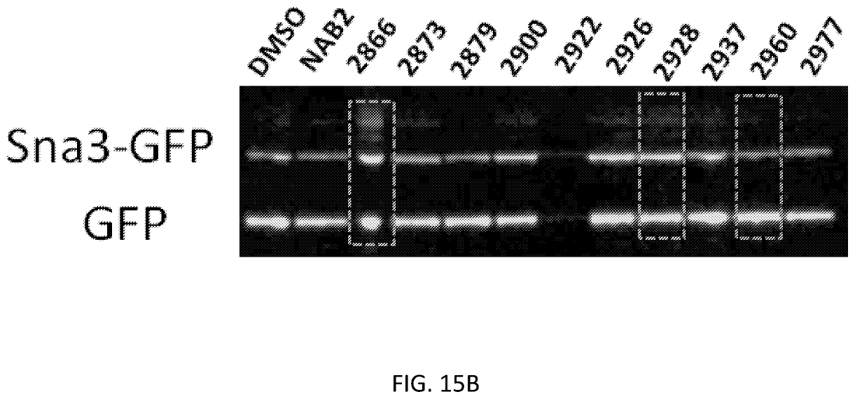

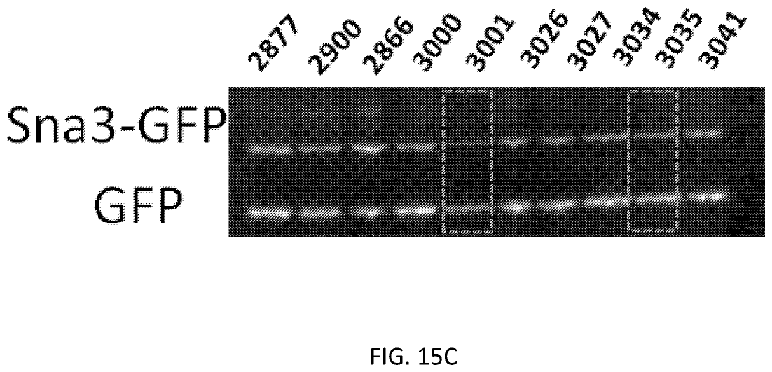

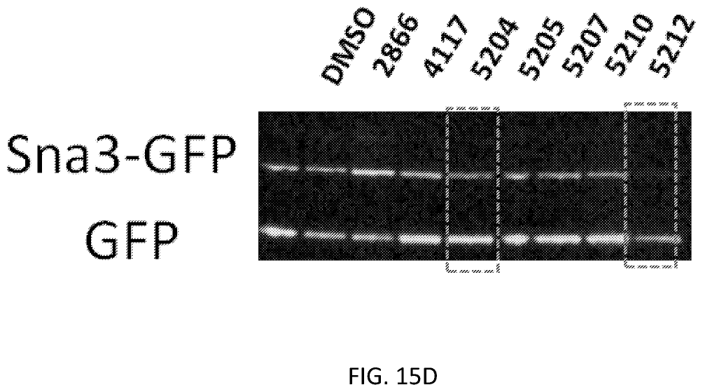

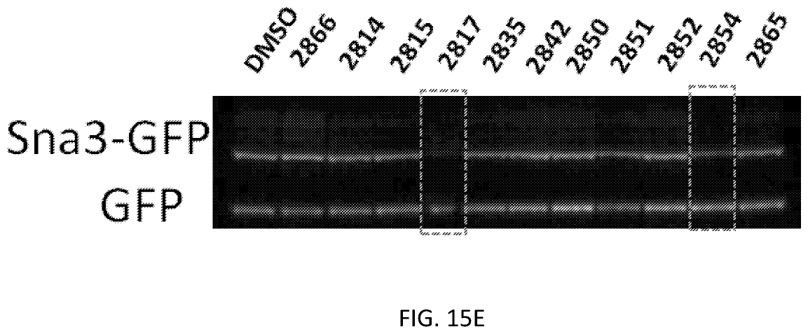

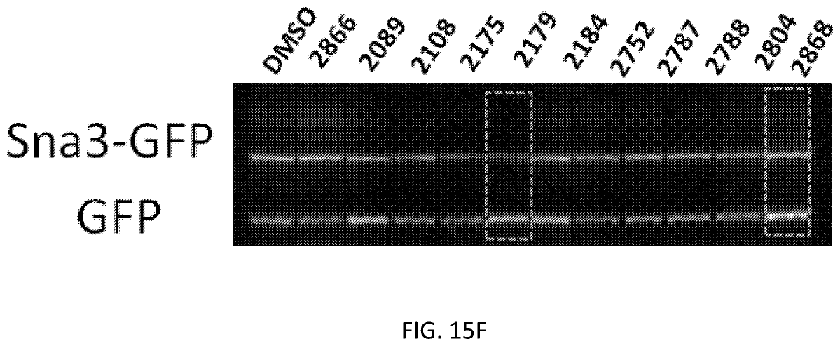

FIG. 15A shows a schematic of Sna3-GFP endosomal trafficking to the vacuole, where GFP is cleaved. Log phase CsmRaf cultures of WT tagged Sna3-GFP cells were shifted to galactose for 5 hours in the presence or absence of the compounds. Cell pellets were lysed in SDS-loading dye and Sna3-GFP cleavage monitored by Western blotting with an anti-GFP antibody. Log phase CsmRaf cultures of WT or α-syn yeast were shifted to galactose for 5 hours in the presence or absence of the compounds at which point they were then prepared for Western blot analysis. FIGS. 15B-15F show Western blot analyses of Sna3-GFP for various compounds. DES-2960 promotes Sna3-GFP trafficking to the vacuole better than DES-2866 and DES-2928 (FIG. 15B). DES-3001 and DES-3035 both promote Sna3-GFP trafficking to the vacuole (FIG. 15C). DES-5204 and DES-5212 both promote Sna3-GFP trafficking to the vacuole (FIG. 15D). DES-2817 and DES-2854 both promote Sna3-GFP trafficking to the vacuole (FIG. 15E). DES-2179 promotes Sna3-GFP trafficking to the vacuole (FIG. 15F).

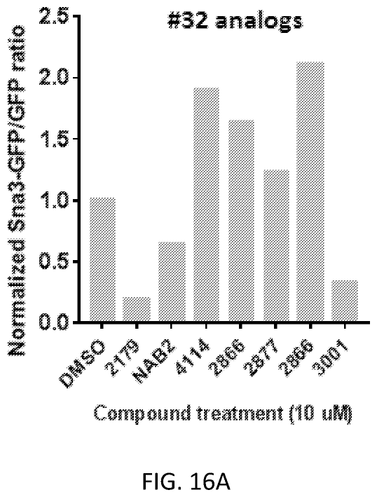

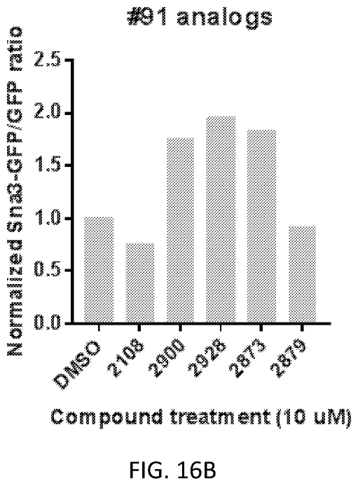

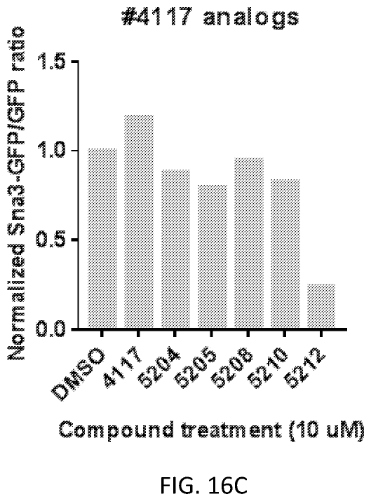

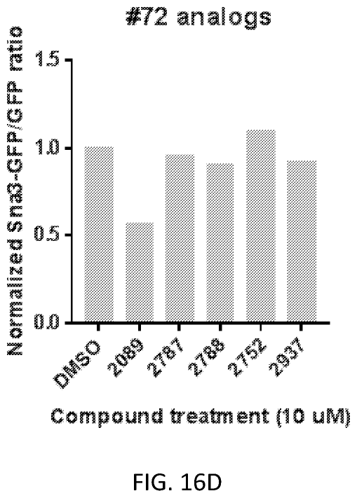

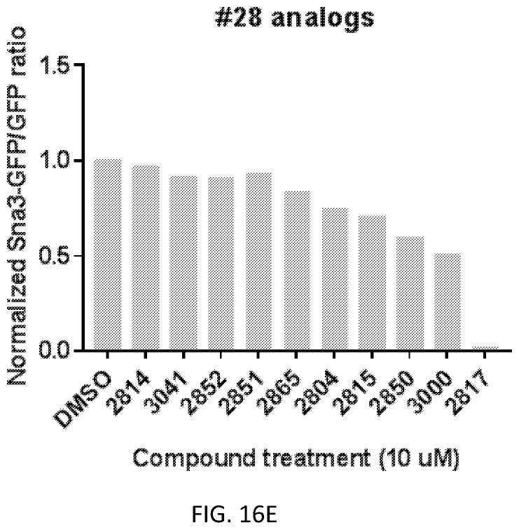

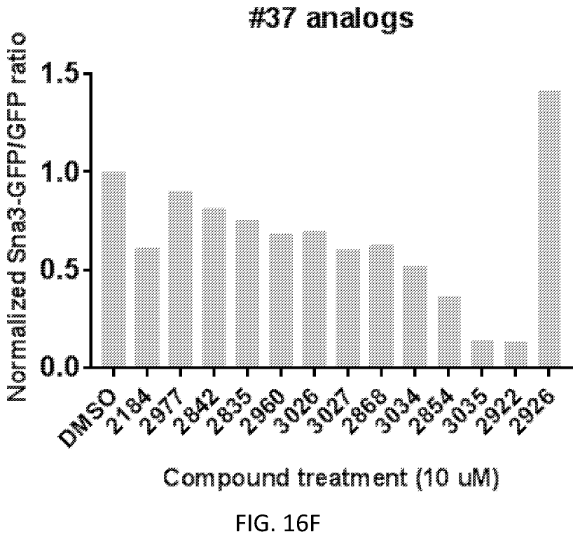

Ratio of the intact Sna3-GFP to cleaved GFP was calculated for each of the conditions as a readout for efficiency of trafficking of the Sna3-GFP molecule to the vacuole. Polyubiquitination of Sna3-GFP was used as a readout for the intermediate step at the multivesicular body. Image Studio software was used to determine the intensities of the bands, based on linear interpolation of the mean signal intensities from each of the areas of interest and ratios were subsequently calculated in Microsoft Excel and plotted according to the compound series as shown in FIGS. 16A-16F. In the ‘32’ series of analogs, the ratio of Sna3-GFP to free GFP varied among the analogs, with DES-2179 having the lowest ratio of Sna3-GFP to free GFP and DES-2866 having the highest ratio (FIG. 16A). In the ‘91’ series of analogs, the ratio of Sna3-GFP to free GFP varied among the analogs, but less so than for the ‘32’ series (FIG. 16B). In the ‘4117’ series of analogs, the ratio of Sna3-GFP to free GFP was lowest for DES-5212 (FIG. 16C). In the ‘72’ series of analogs, the ratio of Sna3-GFP to free GFP was similar for many of the analogs (˜1:1) except for DES-2089, which had a ratio of ˜1:2 (FIG. 16D). In the ‘28’ series of analogs, DES-2817 had the lowest ratio of Sna3-GFP to free GFP (FIG. 16F). The other compounds in the ‘28’ series had ratios between ˜0.5 and 1.0 (FIG. 16F). In the ‘37’ series of analogs, the ratio of Sna3-GFP to free GFP was greatest for DES-2926 (FIG. 16F).

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

Relevant information pertaining to aspects of the present application is also disclosed in the following documents, the contents of which are hereby incorporated by reference:
WO 2014/145887
Tardiff et al., Science 342, 979-983 (2013).
Chung et al., Science 342, 983-987 (2013).
Sims et al., Nature methods 9.3, 303-309 (2012).
Zhang et al., Neuron 78.5, 785-798 (2013).

1. A method for treating neurodegenerative disease in a subject in need thereof, the method comprising administering to the subject an effective amount of a Nedd4 activator of formula (I): 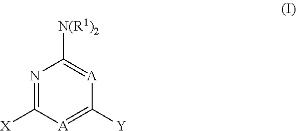
wherein
A is independently CH or N;
R¹ is independently H, (C₁-C₄)-alkyl, phenyl, or each R¹ together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl;
X is 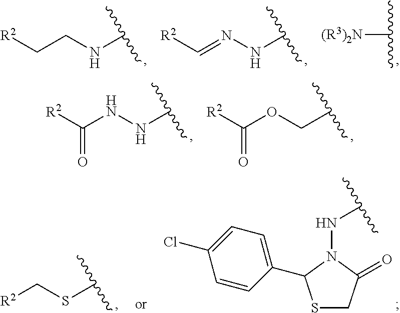
Y is 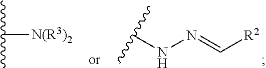
R² is independently phenyl, benzyl, naphthyl, furanyl, indolyl, pyridinyl, pyrazinyl, pyrimidinyl, or thiophenyl, wherein said phenyl, benzyl, naphthyl, furanyl, indolyl, pyridinyl, pyrazinyl, pyrimidinyl, or thiophenyl is optionally independently substituted with one or more H, (C₁-C₄)-alkyl, ((C₁-C₄)-alkyl)OH, OH, O—(C₁-C₄)-alkyl, CF₃, halogen, S—(C₁-C₄)-alkyl, S(O)(C₁-C₄)-alkyl, OC(O)CH₃, OC(O)Ph, OCH₂Ph, OCH₂CO₂H, OCH₂CN, CN, N((C₁-C₄)-alkyl)₂, morpholin-4-yl, or Ph(CO₂H), or is 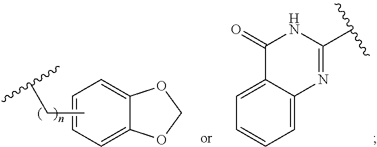
R³ is independently H, (C₁-C₄)-alkyl, phenyl, benzyl, or naphthyl, wherein said phenyl, benzyl, or naphthyl is optionally independently substituted with one or more H, (C₁-C₄)-alkyl, OH, O—(C₁-C₄)-alkyl, CF₃, or halogen, or is (C₁-C₄)-alkyl and each (C₁-C₄)-alkyl together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl, or is 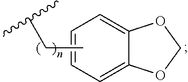
R⁴ is H or (C₁-C₃)-alkyl; and
n is independently 0 or 1.

2. The method of claim 1, wherein the Nedd4 activator is of formula (IA):

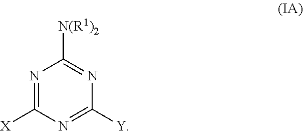

3. The method of claim 1, wherein
X is 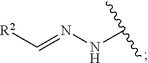
Y is 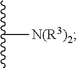
and
R¹ is (C₁-C₄)-alkyl, wherein each R¹ together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl.

4. The method of claim 3, wherein
each R¹ together with the nitrogen to which they are attached form NR⁴-piperazine, piperidine, pyrrolidine, azetidine, or morpholine.

5. The method of claim 4, wherein
each R¹ together with the nitrogen to which they are attached form morpholine.

6. The method of claim 1, wherein
X is 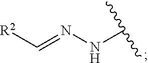
Y is 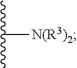
and
R² is phenyl or pyridinyl, wherein said phenyl or pyridinyl is optionally independently substituted with one or more H, (C₁-C₄)-alkyl, OH, O—(C₁-C₄)-alkyl, CF₃, halogen, S—(C₁-C₄)-alkyl, OC(O)CH₃, OC(O)Ph, OCH₂Ph, OCH₂CO₂H, OCH₂CN, CN, N((C₁-C₄)-alkyl)₂, morpholin-4-yl, or Ph(CO₂H).

7. The method of claim 6, wherein
R² is phenyl or pyridine-4-yl, wherein said phenyl or pyridine-4-yl is optionally independently substituted with one or more H, (C₁-C₄)-alkyl, OH, O—(C₁-C₄)-alkyl, CF₃, halogen, OCH₂CN, or N((C₁-C₄)-alkyl)₂.

8. The method of claim 7, wherein
R¹ is (C₁-C₄)-alkyl, wherein each R¹ together with the nitrogen to which they are attached form NR⁴-piperazine, piperidine, pyrrolidine, azetidine, or morpholine.

9. The method of claim 1, wherein
X is 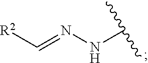
Y is 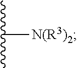
and
R³ is independently H, phenyl, or naphthyl, wherein said phenyl or naphthyl is optionally independently substituted with one or more H, (C₁-C₄)-alkyl, CF₃, or halogen.

10. The method of claim 9, wherein
R² is phenyl or pyridine-4-yl, wherein said phenyl or pyridine-4-yl is optionally independently substituted with one or more H, (C₁-C₄)-alkyl, OH, O—(C₁-C₄)-alkyl, CF₃, halogen, OCH₂CN, or N((C₁-C₄)-alkyl)₂; and
R¹ is (C₁-C₄)-alkyl, wherein each R¹ together with the nitrogen to which they are attached form NR⁴-piperazine, piperidine, pyrrolidine, azetidine, or morpholine.

11. The method of claim 1, wherein
X is 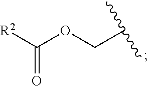
Y is 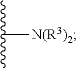
and
R² is phenyl, pyridinyl, or pyrazinyl, wherein said phenyl, pyridinyl, or pyrazinyl, is optionally independently substituted with one or more (C₁-C₄)-alkyl, ((C₁-C₄)-alkyl)OH, OH, O—(C₁-C₄)-alkyl, or S(O)(C₁-C₄)-alkyl.

12. The method of claim 1, wherein
each X and Y is independently

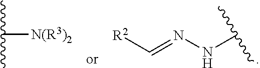

13. (canceled)

14. The method of claim 1, wherein
X is 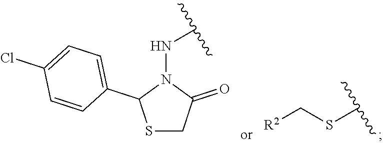
and
R² is

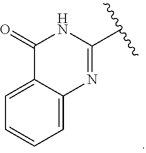 [SMILES: *c1nc2ccccc2c(=O)[nH]1]

15. The method of claim 2, wherein the Nedd4 activator is selected from the group consisting of: 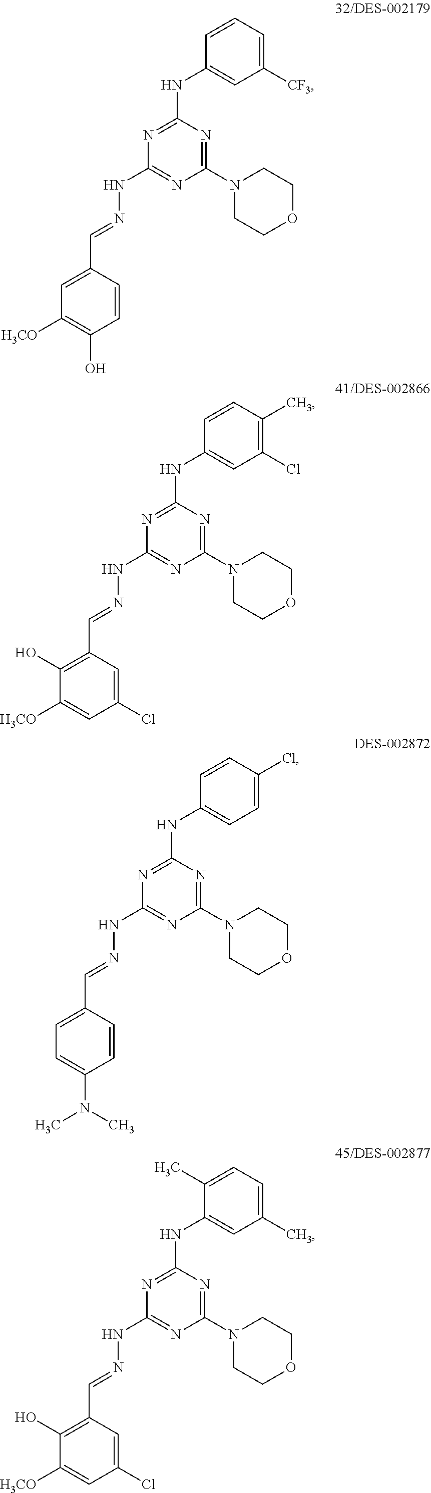 [SMILES: COc1cc(/C=N/Nc2nc(Nc3cccc(C(F)(F)F)c3)nc(N3CCOCC3)n2)ccc1O ; COc1cc(Cl)cc(/C=N/Nc2nc(Nc3ccc(C)c(Cl)c3)nc(N3CCOCC3)n2)c1O ; CN(C)c1ccc(/C=N/Nc2nc(Nc3ccc(Cl)cc3)nc(N3CCOCC3)n2)cc1 ; COc1cc(Cl)cc(/C=N/Nc2nc(Nc3cc(C)ccc3C)nc(N3CCOCC3)n2)c1O] 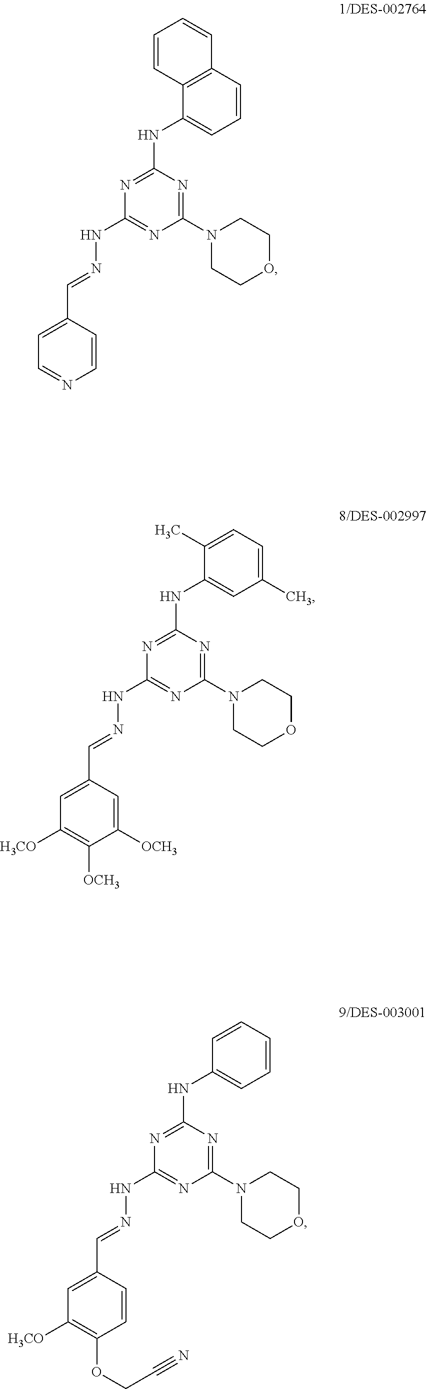 [SMILES: C(=N\Nc1nc(Nc2cccc3ccccc23)nc(N2CCOCC2)n1)/c1ccncc1 ; COc1cc(/C=N/Nc2nc(Nc3cc(C)ccc3C)nc(N3CCOCC3)n2)cc(OC)c1OC ; COc1cc(/C=N/Nc2nc(Nc3ccccc3)nc(N3CCOCC3)n2)ccc1OCC#N] 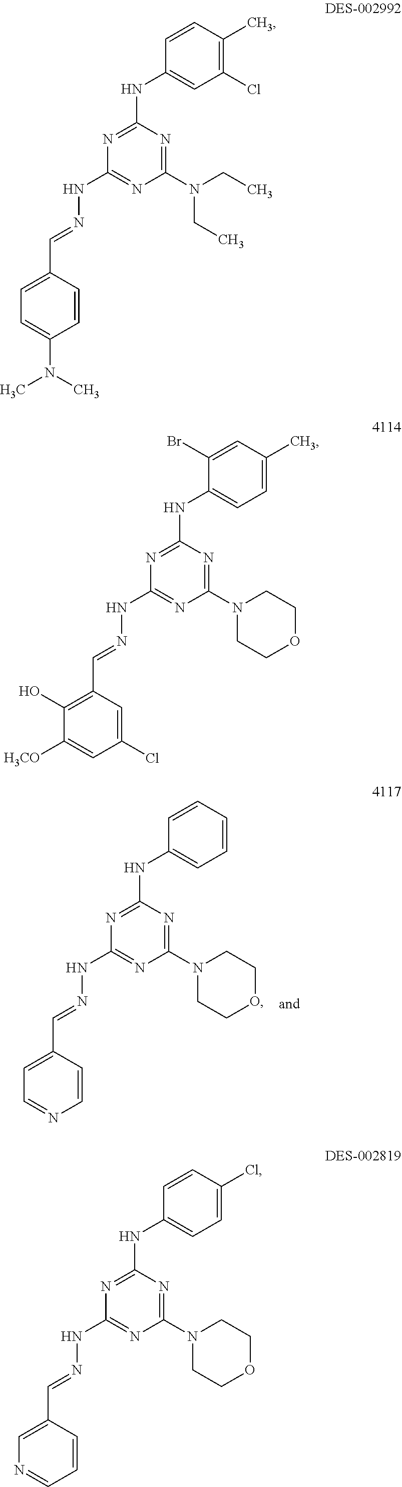 [SMILES: CCN(CC)c1nc(N/N=C/c2ccc(N(C)C)cc2)nc(Nc2ccc(C)c(Cl)c2)n1 ; COc1cc(Cl)cc(/C=N/Nc2nc(Nc3ccc(C)cc3Br)nc(N3CCOCC3)n2)c1O ; C(=N\Nc1nc(Nc2ccccc2)nc(N2CCOCC2)n1)/c1ccncc1 ; Clc1ccc(Nc2nc(N/N=C/c3cccnc3)nc(N3CCOCC3)n2)cc1]
or a pharmaceutically acceptable salt thereof, or a pharmaceutical composition thereof.

16. (canceled)

17. The method of claim 1, wherein the neurodegenerative disease comprises Parkinson's disease, Alzheimer's disease, or Lewy body disease.

18. The method of claim 1, wherein the Nedd4 activator modulates α-synuclein toxicity, modulates ubiquitin mediated endosomal transport, increases ubiquitination or polyubiquitination, modulating E3 ubiquitin ligase, promotes Nedd4 dependent Golgi to vacuole or plasma membrane to vacuole trafficking of adaptor protein Sna3, promotes Nedd4 dependent endocytosis of leucine permease, or any combination thereof.

19. (canceled)

20. (canceled)

21. (canceled)

22. (canceled)

23. (canceled)

24. A method of modulating α-synuclein toxicity in a subject in need thereof, the method comprising administering to the subject an effective amount of a Nedd4 activator of formula (I): 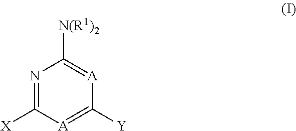
wherein
A is independently Ch or N;
R¹ is independently H, (C₁-C₄)-alkyl, phenyl, or each R¹ together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl;
X is 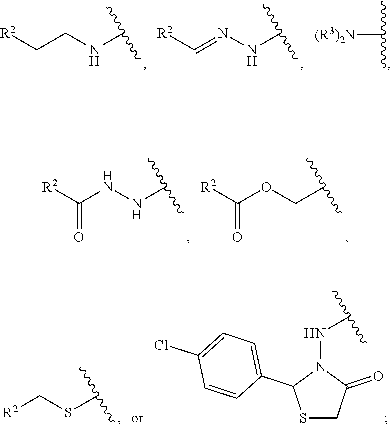
Y is 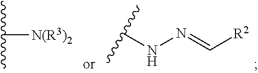
R² is independently phenyl, benzyl, naphthyl, furanyl, indolyl, pyridinyl, pyrazinyl, pyrimidinyl, or thiophenyl, wherein said phenyl, benzyl, naphthyl, furanyl, indolyl, pyridinyl, pyrazinyl, pyrimidinyl, or thiophenyl is optionally independently substituted with one or more H, (C₁-C₄)-alkyl, ((C₁-C₄)-alkyl)OH, OH, O—(C₁-C₄)-alkyl, CF₃, halogen, S—(C₁-C₄)-alkyl, S(O)(C₁-C₄)-alkyl, OC(O)CH₃, OC(O)Ph, OCH₂Ph, OCH₂CO₂H, OCH₂CN, CN, N((C₁-C₄)-alkyl)₂, morpholin-4-yl, or Ph(CO₂H), or is 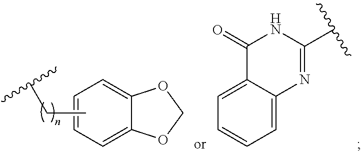
R³ is independently H, (C₁-C₄)-alkyl, phenyl, benzyl, or naphthyl, wherein said phenyl, benzyl, or naphthyl is optionally independently substituted with one or more H, (C₁-C₄)-alkyl, OH, O—(C₁-C₄)-alkyl, CF₃, or halogen, or is (C₁-C₄)-alkyl and each (C₁-C₄)-alkyl together with the nitrogen to which they are attached form a 3-7 membered heterocyclic ring, wherein one of the carbon atoms is optionally replaced with NR⁴, O or S, and wherein the 3-7 membered heterocyclic ring is optionally substituted with a (C₁-C₄)-alkyl, or is 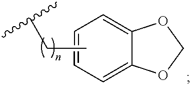
R⁴ is H or (C₁-C₃)-alkyl; and
n is independently 0 or 1.

25. (canceled)

26. (canceled)

27. (canceled)

28. (canceled)

29. (canceled)

30. A method for treating neurodegenerative disease in a subject in need thereof, the method comprising administering to the subject an effective amount of a Nedd4 activator of formula (II): 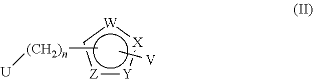
wherein
each of W, X, Y, Z is independently O, S, NR⁶, N, C, or CR⁷;
at least one of W, X, Y, Z must be O, S, NR⁶, or N;
R⁶ is independently H, (C₁-C₃)alkyl, phenyl;
R⁷ is independently H, (C₁-C₃)alkyl, or phenyl;
n is an integer from 0-3;
U is OR⁸, SR⁸, (SO₂)R⁸, (SO₂)NR⁸, N(R⁸)₂, NH(CO)R⁸, NHCH₂R⁸, phenyl, or 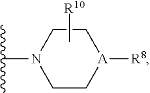
or U is 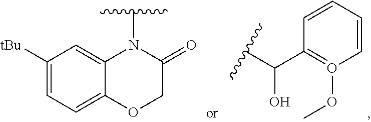
or U is, 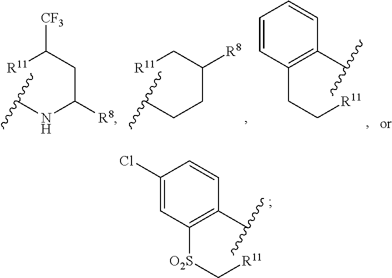
R⁸ is phenyl, naphthyl, pyridinyl, pyrimidinyl, pyrazinyl, quinolinyl, isoquinolinyl or benzothiazolyl, wherein said phenyl, naphthyl, pyridinyl, pyrimidinyl, pyrazinyl, quinolinyl, isoquinolinyl, or benzothiazolyl is optionally independently substituted with one or more H, (C₁-C₄)-alkyl, OH, O—(C₁-C₄)-alkyl, OCF₃, CF₃, halogen, CO₂((C₁-C₄)-alkyl), NH(CO)((C₁-C₄)-alkyl), (C₁-C₄)-alkyl((CO)NH₂), S—(C₁-C₄)-alkyl, triazole, or R⁸ is 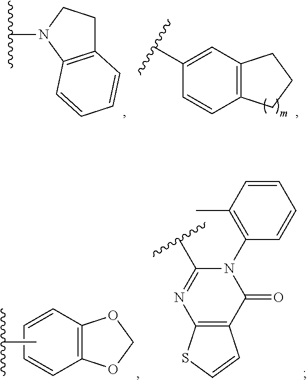
m is 1 or 2;
V is 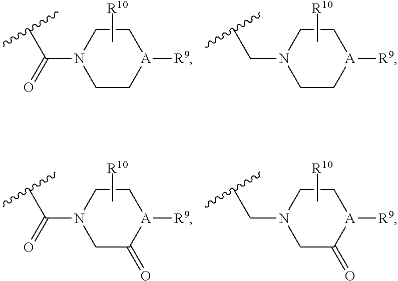
or 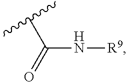
R⁹ is phenyl, pyridinyl, pyrimidinyl, or pyrazinyl, wherein said phenyl, pyridinyl, pyrimidinyl, or pyrazinyl is optionally independently substituted with one or more H, (C₁-C₄)-alkyl, —OH, —O—(C₁-C₄)-alkyl, —CF₃, halogen, —CN, —C(O)((C₁-C₄)-alkyl), or R⁹ is —CH₂CH₂N((C₁-C₄)-alkyl)₂;
A is independently CH, N, or C(OH);
R¹⁰ is H or (C₁-C₄)-alkyl; and
R¹¹ is H or R¹¹ together with the carbon to which it is attached forms a 5-6 membered ring with W or Z.

31. (canceled)

32. (canceled)

33. (canceled)

34. (canceled)

35. The method of claim 18, wherein the Nedd4 activator is:

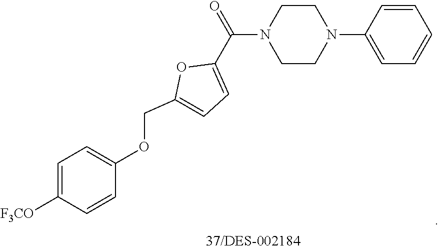 [SMILES: O=C(c1ccc(COc2ccc(OC(F)(F)F)cc2)o1)N1CCN(c2ccccc2)CC1]

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 15, wherein the Nedd4 activator is selected from the group consisting of: 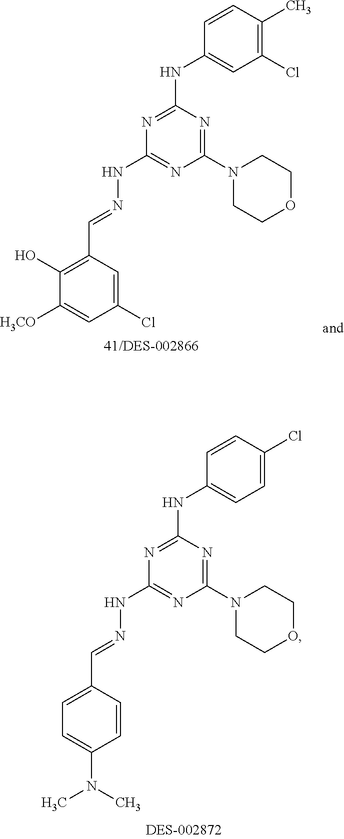 [SMILES: COc1cc(Cl)cc(/C=N/Nc2nc(Nc3ccc(C)c(Cl)c3)nc(N3CCOCC3)n2)c1O ; CN(C)c1ccc(/C=N/Nc2nc(Nc3ccc(Cl)cc3)nc(N3CCOCC3)n2)cc1]
or a pharmaceutically acceptable salt thereof, or a pharmaceutical composition thereof.

56. (canceled)

57. (canceled)

58. (canceled)

59. (canceled)
